SEQUENCE LISTING (22 sequences)

SEQ ID NO: 1 - protein, 7 aa, synthetic construct
GGTFSSY

SEQ ID NO: 2 - protein, 5 aa, synthetic construct
SYFWS

SEQ ID NO: 3 - protein, 10 aa, synthetic construct
GGTFSSYFWS

SEQ ID NO: 4 - protein, 5 aa, synthetic construct
YSGGS

SEQ ID NO: 5 - protein, 16 aa, synthetic construct
RFYSGGSANYNPSLKE

SEQ ID NO: 6 - protein, 11 aa, synthetic construct
ERRGYSGGFEI

SEQ ID NO: 7 - protein, 16 aa, synthetic construct
RSSQSLLHSNSYNYLD

SEQ ID NO: 8 - protein, 7 aa, synthetic construct
LGSYRAS

SEQ ID NO: 9 - protein, 9 aa, synthetic construct
MQPLQTPFT

SEQ ID NO: 10 - protein, 119 aa, synthetic construct
QVQLVQSGAEVKKPGSSVKVSCKASGGTFSSYFWSWVRQAPGQGLEWMGRFYSGGSANYNPSLKERVTITADESTSTAYMELSSLRSEDTAVYYCARERRGYSGGFEIWGQGTLVTVSS

SEQ ID NO: 11 - protein, 112 aa, synthetic construct
DIVMTQSPLSLPVTPGEPASISCRSSQSLLHSNSYNYLDWYLQKPGQSPQLLIYLGSYRASGVPDRFSGSGSGTDFTLKISRVEAEDVGVYYCMQPLQTPFTFGPGTKVDIK

SEQ ID NO: 12 - protein, 676 aa, synthetic construct
QVQLVQSGAEVKKPGSSVKVSCKASGGTFSSYFWSWVRQAPGQGLEWMGRFYSGGSANYNPSLKERVTITADESTSTAYMELSSLRSEDTAVYYCARERRGYSGGFEIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQVQLVQSGAEVKKPGSSVKVSCKASGGTFSSYFWSWVRQAPGQGLEWMGRFYSGGSANYNPSLKERVTITADESTSTAYMELSSLRSEDTAVYYCARERRGYSGGFEIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 219 aa, synthetic construct
DIVMTQSPLSLPVTPGEPASISCRSSQSLLHSNSYNYLDWYLQKPGQSPQLLIYLGSYRASGVPDRFSGSGSGTDFTLKISRVEAEDVGVYYCMQPLQTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, synthetic construct
QVQLVQSGAEVKKPGSSVKVSCKASGGTFSSYFWSWVRQAPGQGLEWMGRFYSGGSANYNPSLKERVTITADESTSTAYMELSSLRSEDTAVYYCARERRGYSGGFEIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 16 - protein, 415 aa, Mus musculus
MRLPRASSPCGLAWGPLLLGLSGLLVASQPQLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLLLAILLSLVLFLLFTTVLACAWMRHPSLCRKLGTLLKRHPEGEESPPCPAPRADPHFPDLAEPLLPMSGDLSPSPAGPPTAPSLEEVVLQQQSPLVQARELEAEPGEHGQVAHGANGIHVTGGSVTVTGNIYIYNGPVLGGTRGPGDPPAPPEPPYPTPEEGAPGPSELSTPYQEDGKAWHLAETETLGCQDL

SEQ ID NO: 17 - protein, 440 aa, Macaca fascicularis
MRLPWATSAPGLAWGPLVLGLFGLLAASQPQVVRKGPVPPYGSENQTCRDQEKEYYEPRHRICCSRCPPGTYVSAKCSRSRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCEDQGLVEAAPGTAQSDTTCRNPSESLPPEMSGTMLMLAILLPLAFFLLLATIFACIWKSHPSLCRKLGSLLKRHPQGEGPNPVAAGRDPPKANPQYPDLVEPLLPISGDVSPVSTGLPTALVSEEGVPQQQSPLDLTTEPQLEPGEQNQVAHGTNGIHVTGGSMTITGNIYIYNGPVLGGPPGPGDLPATPDPPYPIPEEGDPGPPGLSTPHQEDGKAWHLAETEHCGATPSNRGPRSQFITYD

SEQ ID NO: 18 - protein, 10 aa, synthetic construct
  REGION 1..10: note = Synthetic
EPKSCGGGGS

SEQ ID NO: 19 - DNA, 2028 bp, Synthetic construct
  source 1..2028: mol_type = other DNA
caagtgcagcttgtgcagagcggagccgaagtcaagaagcccgggtcgtcagtgaaagtgtcctgcaaggcctccggcggaaccttcagctcgtacttctggtcctgggttaggcaggcccctggacaaggcctcgaatggatgggtcggttctactccggcggttccgccaactacaacccgtcactgaaggagagagtgaccattaccgcggatgagtcgactagcaccgcctacatggaactgtccagcctgcggtccgaggacactgcagtctactattgtgctcgcgagcggcgcgggtactctggcggatttgaaatctggggacagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcctaagagctgcggtggcggtggatcacaagtgcagcttgtgcagagcggagccgaagtcaagaagcccgggtcgtcagtgaaagtgtcctgcaaggcctccggcggaaccttcagctcgtacttctggtcctgggttaggcaggcccctggacaaggcctcgaatggatgggtcggttctactccggcggttccgccaactacaacccgtcactgaaggagagagtgaccattaccgcggatgagtcgactagcaccgcctacatggaactgtccagcctgcggtccgaggacactgcagtctactattgtgctcgcgagcggcgcgggtactctggcggatttgaaatctggggacagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtcccccggaaaa

SEQ ID NO: 20 - DNA, 657 bp, synthetic construct
  source 1..657: mol_type = other DNA
gacattgtgatgactcagtcgccgttgtcgctgcctgtgactcctggagagccggcctcgatctcctgccggtcaagccagtccctgctgcactccaactcctataactacctggattggtacctccaaaagcctgggcagagcccccagctcctgatctaccttggctcttaccgggcctccggagtgccggacagattcagcggttccggatcaggaaccgactttaccctgaaaatctcccgcgtggaagcggaagatgtcggcgtgtactactgtatgcagcccctgcaaactccattcaccttcgggcccggcaccaaggtcgacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 21 - DNA, 357 bp, Synthetic construct
  source 1..357: mol_type = other DNA
caagtgcagcttgtgcagagcggagccgaagtcaagaagcccgggtcgtcagtgaaagtgtcctgcaaggcctccggcggaaccttcagctcgtacttctggtcctgggttaggcaggcccctggacaaggcctcgaatggatgggtcggttctactccggcggttccgccaactacaacccgtcactgaaggagagagtgaccattaccgcggatgagtcgactagcaccgcctacatggaactgtccagcctgcggtccgaggacactgcagtctactattgtgctcgcgagcggcgcgggtactctggcggatttgaaatctggggacagggaaccctggtcaccgtctcctca

SEQ ID NO: 22 - DNA, 336 bp, synthetic construct
  source 1..336: mol_type = other DNA
gacattgtgatgactcagtcgccgttgtcgctgcctgtgactcctggagagccggcctcgatctcctgccggtcaagccagtccctgctgcactccaactcctataactacctggattggtacctccaaaagcctgggcagagcccccagctcctgatctaccttggctcttaccgggcctccggagtgccggacagattcagcggttccggatcaggaaccgactttaccctgaaaatctcccgcgtggaagcggaagatgtcggcgtgtactactgtatgcagcccctgcaaactccattcaccttcgggcccggcaccaaggtcgacatcaaa

(19) United States
(12) Patent Application Publication
(10) Pub. No.: US 2023/0365701 A1
(43) Pub. Date: Nov. 16, 2023

(54) ANTI-LYMPHOTOXIN BETA RECEPTOR ANTIBODIES AND METHODS OF USE THEREOF

(71) Applicant: Pfizer Inc., New York, NY (US)

(72) Inventors: James Reasoner APGAR, Newton, MA (US); Bas Joannes Gertrudis BAATEN, San Diego, CA (US); Aruna BITRA, San Diego, CA (US); Javier Fernando CHAPARRO RIGGERS, San Diego, CA (US); Giuseppe DI CARO, La Jolla, CA (US); Pawel Kamil DOMINIK, San Diego, CA (US); Zachary John MABEN, Winchester, MA (US); Lidia MOSYAK, Newton, MA (US); Andrew Ross NAGER, San Diego, CA (US); Cecilia Marianne ODERUP, Malmö (SE); Edward Derrick PASCUA, San Diego, CA (US); Shahram SALEK-ARDAKANI, La Jolla, CA (US); Dirk Michael ZAJONC, Poway, CA (US)

(73) Assignee: Pfizer Inc., New York, NY (US)

(21) Appl. No.: 18/315,054

(22) Filed: May 10, 2023

Related U.S. Application Data
(60) Provisional application No. 63/340,753, filed on May 11, 2022.

(51) Int. Cl. C07K 16/28; A61P 35/00

(52) U.S. Cl. CPC C07K 16/2878; A61P 35/00; C07K 2317/35; C07K 2317/71; C07K 2317/92; A61K 2039/505

(57) ABSTRACT

Antibodies that specifically bind to LTBR are provided. Also provided are uses of these antibodies, and related compositions and methods.

### REFERENCE TO SEQUENCE LISTING

The instant application contains a Sequence Listing which has been submitted electronically in .xml format and is hereby incorporated by reference in its entirety. Said Sequence Listing, created on Mar. 21, 2023, is named PC072526A_SeqListing_ST.26.xml and is 27 kilobytes in size.

### BACKGROUND

The present invention relates to antibodies that specifically bind to lymphotoxin beta receptor (LTBR)(also known as Tumor Necrosis Factor Receptor superfamily member 3). The present invention also pertains to related molecules, e.g. nucleic acids which encode such antibodies, compositions, and related methods, e.g., methods for producing and purifying such antibodies, and their use in diagnostics and therapeutics.

LTBR is a widely expressed tumor necrosis factor receptor (TNFR) family member that plays an important role in the development and organization of lymphoid cells. The natural ligands for LTBR are LIGHT and LTalpha1beta2 (LTa1B2) heterotrimer. LTBR has pleiotropic functions and plays a central role in the formation and structure of secondary lymphoid organs (i.e. lymph nodes) and tertiary lymphoid structure (TLS) during homeostasis or inflammatory conditions. Activation of LTBR on stromal cells leads to the expression of adhesion molecules, the production of homeostatic chemokines, and the differentiation of endothelial cells to high endothelial venules that collectively result in the structural organization of lymphoid tissue and the recruitment of naïve CD8+ T cells. Moreover, LTBR stimulation on DCs may enhance their function resulting in the expansion of effector CD8+ T cells.

The presence of TLS in human tumors is associated with enhanced anti-tumor immunity and improved prognosis, particularly in anti-PD1 or anti-PDL1 [PD(L)1] treated patients (Sautes-Fridman, C et al, Nat Rev Cancer 2019 June, 19(6): 307-325; Cabrita R et al, Nature 2020 January, 577(7791): 561-565; Helmink B et al, Nature 2020 January, 577(7791): 549-555). Thus, targeting LTBR may have important clinical implications for cancer immunotherapy. This hypothesis is supported by preclinical reports demonstrating that agonism of LTBR promotes TLS formation, enhances T cell infiltration, and overcomes resistance to treatment with anti-PD(L)1 agents in mouse tumor models (Schrama D et al, Immunity 2001 February, 14(2): 111-21; Lukashev, M, et al, Cancer Res 2006 October, 66 (19); Schrama D et al, Cancer Immunol Immunother 2008 January, 57 (1): 85-95; Johansson-Percival A et al, Nat Immunol 2017 November, 18 (11): 1207-1217; Tang H et al, Cancer Cell 2016 March, 14 29(3): 285-296; Allen E et al, Sci Transl Med, 2017 Apr. 12, 9(385): eaak9769), suggesting that LTβR agonism can induce de novo intra-tumoral CD8+ T cell activation.

While LTBR is a potential target for treating cancer and other conditions, improved anti-LTBR antibodies are needed.

### SUMMARY

The present disclosure provides antibodies that bind to lymphotoxin beta receptor (LTBR) as well as uses of the antibodies and associated methods. The disclosure also provides processes for making, preparing, and producing antibodies that bind to LTBR. Antibodies of the disclosure are useful in one or more of diagnosis, prophylaxis, or treatment of disorders or conditions mediated by, associated with, or inhibited by LTBR activity, including, but not limited to cancer. In some embodiments, the antibodies provided herein are agonist antibodies that activate LTBR and associated downstream effects. The disclosure further encompasses expression of antibodies, and preparation and manufacture of compositions comprising antibodies of the disclosure, such as medicaments for the use of the antibodies.

Polynucleotides encoding antibodies that bind LTBR are provided. For example, polynucleotides encoding antibody heavy chains or light chains, or both are also provided. Host cells that express the antibodies are provided. Methods of treatment using the antibodies are provided. Such methods include, but are not limited to, one or more of methods of treating or methods of preventing diseases associated with, mediated by, or inhibited by LTBR expression and or LTBR activation, such as cancer.

In some embodiments, provided herein is an isolated antibody that binds to lymphotoxin beta receptor (LTBR) and comprises a heavy chain variable region (VH) and a light chain variable region (VL), wherein the VH complementarity determining region (CDR) one comprises the amino acid sequence of SEQ ID NOs: 1, 2, or 3, the VH CDR2 comprises the amino acid sequence of SEQ ID NOs: 4 or 5, the VH CDR3 comprises the amino acid sequence of SEQ ID NO: 6, the VL CDR1 comprises the amino acid sequence of SEQ ID NO: 7, the VL CDR2 comprises the amino acid sequence of SEQ ID NO: 8, and the VL CDR3 comprises the amino acid sequence of SEQ ID NO: 9.

In some embodiments, provided herein is a portion of an isolated antibody, wherein the isolated antibody binds to LTBR and comprises a heavy chain variable region (VH) and a light chain variable region (VL), wherein the VH CDR1 comprises the amino acid sequence of SEQ ID NOs: 1, 2, or 3, the VH CDR2 comprises the amino acid sequence of SEQ ID NOs: 4 or 5, the VH CDR3 comprises the amino acid sequence of SEQ ID NO: 6, the VL CDR1 comprises the amino acid sequence of SEQ ID NO: 7, the VL CDR2 comprises the amino acid sequence of SEQ ID NO: 8, and the VL CDR3 comprises the amino acid sequence of SEQ ID NO: 9, wherein the portion binds to LTBR.

In some embodiments, provided herein is an isolated antibody that binds to LTBR comprising: a VH amino acid sequence comprising a VH CDR1, VH CDR2, and VH CDR3 of the amino acid sequence of SEQ ID NO: 10 and a VL amino acid sequence comprising a VL CDR1, VL CDR2, and VL CDR3 of the amino acid sequence of SEQ ID NO: 11.

In some embodiments, provided herein is an antibody comprising a VH of SEQ ID NO: 10 and a VL of SEQ ID NO: 11.

In some embodiments, provided herein is an isolated antibody comprising a light chain comprising the amino acid sequence of SEQ ID NO: 13 and a heavy chain comprising the amino acid sequence of SEQ ID NO: 14, wherein the C-terminal lysine of SEQ ID NO:14 is optional.

In some embodiments, provided herein is an isolated tetravalent antibody comprising a first antigen binding site, a second antigen binding site, a third antigen binding site, and a fourth antigen binding site, which each of the first, second, third, and fourth antigen binding sites bind to LTBR, and wherein each of the first, second, third, and fourth antigen binding sites comprise a VH and VL, and wherein for each of the first, second, third, and fourth antigen binding sites VH and VL: the VH complementarity determining region (CDR) one comprises the amino acid sequence of SEQ ID NOs: 1, 2, or 3, the VH CDR2 comprises the amino acid sequence of SEQ ID NOs: 4 or 5, the VH CDR3 comprises the amino acid sequence of SEQ ID NO: 6, the VL CDR1 comprises the amino acid sequence of SEQ ID NO: 7, the VL CDR2 comprises the amino acid sequence of SEQ ID NO: 8, and the VL CDR3 comprises the amino acid sequence of SEQ ID NO: 9.

In some embodiments, provided herein is an isolated antibody comprising a light chain comprising the amino acid sequence of SEQ ID NO: 13 and a heavy chain comprising the amino acid sequence of SEQ ID NO: 12, wherein the C-terminal lysine of SEQ ID NO:12 is optional.

In some embodiments, provided herein is an isolated polynucleotide or polynucleotides comprising one or more nucleotide sequences encoding the VH, VL, or both of an antibody that binds LTBR, wherein the polynucleotide(s) comprise the VH nucleic acid sequence of SEQ ID NO: 21, the VL nucleic acid sequence of SEQ ID NO: 22, or both the VH nucleic acid sequence of SEQ ID NO: 21 and the VL nucleic acid sequence of SEQ ID NO: 22.

In some embodiments, provided herein is an isolated polynucleotide or polynucleotides comprising one or more nucleotide sequences encoding the heavy chain, light chain, or both of an antibody that binds LTBR, wherein the polynucleotide(s) comprise the heavy chain nucleic acid sequence of SEQ ID NO: 19, the light chain nucleic acid sequence of SEQ ID NO: 20, or both the heavy chain nucleic acid sequence of SEQ ID NO: 19 and the light chain nucleic acid sequence of SEQ ID NO: 20.

In some embodiments, provided herein is an isolated polynucleotide or polynucleotides comprising one or more nucleotide sequences encoding the VH, VL, or both of an antibody that binds LTBR, wherein the polynucleotide(s) comprise the VH nucleic acid sequence of the insert of the plasmid deposited with the ATCC and having ATCC Accession Number PTA-127515, the VL nucleic acid sequence of the insert of the plasmid deposited with the ATCC and having ATCC Accession Number PTA-127516, or both the VH nucleic acid sequence of the insert of the plasmid deposited with the ATCC and having ATCC Accession Number PTA-127515 and the VL nucleic acid sequence of the insert of the plasmid deposited with the ATCC and having ATCC Accession Number PTA-127516.

In some embodiments, provided herein is an isolated antibody that binds to LTBR, wherein the antibody binds to an epitope on LTBR comprising one or more amino acid residues selected from the group consisting of P63, P64, G65, T66, Y67, S69, A70, R76, T78, V79, C80, T82, C83, A84, E85, W92, K119 of SEQ ID NO: 15.

In some embodiments, provided herein is an isolated antibody that binds to human LTBR, wherein the antibody is tetravalent and comprises a first antigen binding site, a second antigen binding site, a third antigen binding site, and a fourth antigen binding site, and wherein each of the first, second, third, and fourth antigen binding sites bind to the same epitope on four different LTBR molecules, and wherein the epitope is on the first cysteine rich domain (CRD1), the second cysteine rich domain (CRD2), or on a combination of CRD1 and CRD2 of LTBR.

### BRIEF DESCRIPTION OF THE DRAWINGS

FIG. 1A shows a schematic of a bivalent anti-LTBR antibody provided herein. The two light chains show the light chain variable (VL) and constant (CK) domains. The two heavy chains show the heavy chain variable (VH) and constant (CH1, CH2, and CH3) domains. FIG. 1B shows a schematic of a tetravalent anti-LTBR antibody provided herein. The four light chains show the light chain variable (VL) and constant (CK) domains. The two heavy chains show the heavy chain variable (VH) and constant (CH1, CH2, and CH3) domains. FIG. 1C shows a schematic of exemplary clustering of 4 LTBR molecules by a tetravalent anti-LTBR antibody provided herein, and exemplary NFkB activation and downstream signaling.

FIGS. 2A-2D show the results of a panel of assays comparing the activity of bivalent vs tetravalent anti-LTBR antibodies. In each of FIGS. 2A and 2D, the data points for the tetravalent antibody are depicted with an inverted triangle and for the bivalent antibody are depicted with a square. In FIG. 2B, the data points for the tetravalent antibody are depicted with an inverted triangle and for the bivalent antibody are depicted with a circle. In FIG. 2C, the X-axis is labeled with the different antibodies. In FIGS. 2A, 2B, and 2D, a range of concentrations of each antibody was used as shown on the X-axis. FIG. 2A shows NFkB activation in a reporter system in HEK293 cells; FIG. 2B shows CXCL10 induction in primary human fibroblasts; FIG. 2C shows IL-12 induction in primary human monocyte-derived dendritic cells (moDC); FIG. 2D shows CXCL10 induction in primary cynomolgus monkey fibroblasts.

FIG. 3 shows a surface representation of the binding epitope of 5A7 antibody on mouse LTBR (FIG. 3, left side) and 1E01 antibody on human LTBR (FIG. 3, right side). Residues of mouse LTBR or human LTBR participating in the interaction interface with 5A7 or 1E01 are shaded on the left or right side model, respectively. Residues of the shared epitope between mouse and human LTBR involving in binding to 5A7 and 1E01, respectively, are circled.

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:

E1. An isolated antibody that binds to lymphotoxin beta receptor (LTBR) and comprises a heavy chain variable region (VH) and a light chain variable region (VL), wherein the VH complementarity determining region (CDR) one comprises the amino acid sequence of SEQ ID NOs: 1, 2, or 3, the VH CDR2 comprises the amino acid sequence of SEQ ID NOs: 4 or 5, the VH CDR3 comprises the amino acid sequence of SEQ ID NO: 6, the VL CDR1 comprises the amino acid sequence of SEQ ID NO: 7, the VL CDR2 comprises the amino acid sequence of SEQ ID NO: 8, and the VL CDR3 comprises the amino acid sequence of SEQ ID NO: 9.

E2. The antibody of E1, wherein the VH comprises the amino acid sequence of SEQ ID NO: 10 or a variant of SEQ ID NO: 10 thereof comprising one to four amino acid substitutions at residues that are not within a CDR, and the VL comprises the amino acid sequence of SEQ ID NO: 11 or a variant of SEQ ID NO: 11 thereof comprising one to four amino acid substitutions at residues that are not within a CDR.

E3. The antibody of E2, wherein the VH comprises the amino acid sequence of SEQ ID NO: 10 and the VL comprises the amino acid sequence of SEQ ID NO: 11.

E4. A portion of an isolated antibody, wherein the isolated antibody binds to LTBR and comprises a heavy chain variable region (VH) and a light chain variable region (VL), wherein the VH CDR1 comprises the amino acid sequence of SEQ ID NOs: 1, 2, or 3, the VH CDR2 comprises the amino acid sequence of SEQ ID NOs: 4 or 5, the VH CDR3 comprises the amino acid sequence of SEQ ID NO: 6, the VL CDR1 comprises the amino acid sequence of SEQ ID NO: 7, the VL CDR2 comprises the amino acid sequence of SEQ ID NO: 8, and the VL CDR3 comprises the amino acid sequence of SEQ ID NO: 9, wherein the portion binds to LTBR.

E5. An isolated antibody that binds to LTBR comprising: a VH amino acid sequence comprising a VH CDR1, VH CDR2, and VH CDR3 of the amino acid sequence of SEQ ID NO: 10 and a VL amino acid sequence comprising a VL CDR1, VL CDR2, and VL CDR3 of the amino acid sequence of SEQ ID NO: 11.

E6. An isolated antibody comprising a light chain comprising the amino acid sequence of SEQ ID NO: 13 and a heavy chain comprising the amino acid sequence of SEQ ID NO: 14, wherein the C-terminal lysine of SEQ ID NO:14 is optional.

E7. An isolated tetravalent antibody comprising a first antigen binding site, a second antigen binding site, a third antigen binding site, and a fourth antigen binding site, which each of the first, second, third, and fourth antigen binding sites bind to LTBR, and wherein each of the first, second, third, and fourth antigen binding sites comprise a VH and VL, and wherein for each of the first, second, third, and fourth antigen binding sites VH and VL; the VH complementarity determining region (CDR) one comprises the amino acid sequence of SEQ ID NOs: 1, 2, or 3, the VH CDR2 comprises the amino acid sequence of SEQ ID NOs: 4 or 5, the VH CDR3 comprises the amino acid sequence of SEQ ID NO: 6, the VL CDR1 comprises the amino acid sequence of SEQ ID NO: 7, the VL CDR2 comprises the amino acid sequence of SEQ ID NO: 8, and the VL CDR3 comprises the amino acid sequence of SEQ ID NO: 9.

E8. The antibody of E7, wherein each of the first, second, third, and fourth VH comprises the amino acid sequence of SEQ ID NO: 10 or a variant of SEQ ID NO: 10 thereof comprising one to four amino acid substitutions at residues that are not within a CDR, and each of the first, second, third, and fourth VL comprises the amino acid sequence of SEQ ID NO: 11 or a variant of SEQ ID NO: 11 thereof comprising one to four amino acid substitutions at residues that are not within a CDR.

E9. The antibody of E8, wherein each of the first, second, third, and fourth VH comprises the amino acid sequence of SEQ ID NO: 10 and each of the first, second, third, and fourth VL comprises the amino acid sequence of SEQ ID NO: 11.

E10. An isolated antibody comprising a light chain comprising the amino acid sequence of SEQ ID NO: 13 and a heavy chain comprising the amino acid sequence of SEQ ID NO: 12, wherein the C-terminal lysine of SEQ ID NO:12 is optional.

E11. The antibody of E10, wherein the antibody comprises four light chains and two heavy chains.

E12. The antibody of any one of E10 and E11, wherein the antibody is capable of simultaneously binding four separate LTBR molecules.

E13. An isolated antibody that binds to LTBR, wherein the antibody binds to an epitope on LTBR comprising one or more amino acid residues selected from the group consisting of P63, P64, G65, T66, Y67, S69, A70, R76, T78, V79, C80, T82, C83, A84, E85, W92, K119 of SEQ ID NO: 15.

E14. The antibody of E13, wherein the antibody binds to an epitope on LTBR comprising two, three, four, five, six, seven, eight, nine, ten, or all of the amino acid residues selected from the group consisting of P63, P64, G65, T66, Y67, S69, A70, R76, T78, V79, C80, T82, C83, A84, E85, W92, K119 of SEQ ID NO: 15.

E15. The antibody of any one or more of E13 and E14, wherein the antibody is tetravalent and comprises a first antigen binding site, a second antigen binding site, a third antigen binding site, and a fourth antigen binding site, and wherein each of the first, second, third, and fourth antigen binding sites bind to the same epitope on four different LTBR molecules.

E16. An isolated antibody that binds to human LTBR, wherein the antibody is tetravalent and comprises a first antigen binding site, a second antigen binding site, a third antigen binding site, and a fourth antigen binding site, and wherein each of the first, second, third, and fourth antigen binding sites bind to the same epitope on four different LTBR molecules, and wherein the epitope is on the first cysteine rich domain (CRD1), the second cysteine rich domain (CRD2), or on a combination of CRD1 and CRD2 of LTBR.

E17. The antibody of any one of E1-E16, wherein the antibody mediates at least one of the following activities: (i) promotes the clustering of LTBR on the cell membrane; (ii) promotes LTBR-mediated NFkB activation; (iii) induces Cxcl10 secretion; (iv) induces IL-12 secretion; (v) inhibits tumor growth.

E18. The antibody of any one of E1-E17, wherein the antibody does not block the binding of ligand to LTBR.

E19. The antibody of 18, wherein the ligand is LIGHT or LTa1b2.

E20. The antibody of any one of E13-E19, wherein the antibody comprises a heavy chain variable region (VH), and wherein the VH complementarity determining region (CDR) one comprises the amino acid sequence of SEQ ID NOs: 1, 2, or 3, the VH CDR2 comprises the amino acid sequence of SEQ ID NOs: 4 or 5, and the VH CDR3 comprises the amino acid sequence of SEQ ID NO: 6.

E21. The antibody of any one of E13-E20, wherein the antibody comprises a light chain variable region (VL), and wherein the VL CDR1 comprises the amino acid sequence of SEQ ID NO: 7, the VL CDR2 comprises the amino acid sequence of SEQ ID NO: 8, and the VL CDR3 comprises the amino acid sequence of SEQ ID NO: 9.

E22. The antibody of any one of E13-E21, wherein the antibody comprises a VH that comprises the amino acid sequence of SEQ ID NO: 10 and the antibody comprises a VL that comprises the amino acid sequence of SEQ ID NO: 11.

E23. The antibody of any one of E1-E22, wherein the antibody is tetravalent and has modifications in the Fc domain to reduce binding to the Fc gamma receptor, optionally wherein the modifications are L234A, L235A, and G237A (EU numbering).

E24. An isolated polynucleotide or polynucleotides comprising one or more nucleotide sequences encoding the antibody of any one of E1-E23.

E25. An isolated polynucleotide or polynucleotides comprising one or more nucleotide sequences encoding the VH, VL, or both of an antibody that binds LTBR, wherein the polynucleotide(s) comprise the VH nucleic acid sequence of SEQ ID NO: 21, the VL nucleic acid sequence of SEQ ID NO: 22, or both the VH nucleic acid sequence of SEQ ID NO: 21 and the VL nucleic acid sequence of SEQ ID NO: 22.

E26. An isolated polynucleotide or polynucleotides comprising one or more nucleotide sequences encoding the heavy chain, light chain, or both of an antibody that binds LTBR, wherein the polynucleotide(s) comprise the heavy chain nucleic acid sequence of SEQ ID NO: 19, the light chain nucleic acid sequence of SEQ ID NO: 20, or both the heavy chain nucleic acid sequence of SEQ ID NO: 19 and the light chain nucleic acid sequence of SEQ ID NO: 20.

E27. An isolated polynucleotide or polynucleotides comprising one or more nucleotide sequences encoding the VH, VL, or both of an antibody that binds LTBR, wherein the polynucleotide(s) comprise the VH nucleic acid sequence of the insert of the plasmid deposited with the ATCC and having ATCC Accession Number PTA-127515, the VL nucleic acid sequence of the insert of the plasmid deposited with the ATCC and having ATCC Accession Number PTA-127516, or both the VH nucleic acid sequence of the insert of the plasmid deposited with the ATCC and having ATCC Accession Number PTA-127515 and the VL nucleic acid sequence of the insert of the plasmid deposited with the ATCC and having ATCC Accession Number PTA-127516.

E28. The polynucleotide or polynucleotides of any one of E24-E27, wherein said polynucleotide(s) is RNA or DNA.

E29. The polynucleotide or polynucleotides of any one of E24-E28, wherein said polynucleotide(s) comprises at least one chemical modification.

E30. The polynucleotide or polynucleotides of E29, wherein the chemical modification wherein is selected from pseudouridine, 1-methylpseudouridine. N1-methylpseudouridine, 35 N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine), 5-methoxyuridine and 2′-O-methyl uridine.

E31. The polynucleotide or polynucleotides of any one of E24-E28, wherein said polynucleotide does not comprise a chemical modification.

E32. A vector comprising the polynucleotide or polynucleotides of any one of E24-E31.

E33. An isolated host cell comprising the polynucleotide or polynucleotides of any one of E24-E31 or the vector of E32.

E34. A method of producing an antibody, comprising culturing the host cell of E33 under conditions that result in production of the antibody, and recovering the antibody.

E35. A pharmaceutical composition comprising a therapeutically effective amount of the antibody of any one of E1-E23 and a pharmaceutically acceptable carrier.

E36. A method of treating cancer in a subject, the method comprising administering to a subject in need thereof a therapeutically effective amount of the pharmaceutical composition of claim 35 or the antibody of any one of E1-E23.

E37. The antibody of any one of E1-E23 for use a medicament.

E38. The antibody of E37 wherein the medicament is for the treatment of cancer.

E39. The antibody of any one of E1-E23 for use for the treatment of cancer.

E40. The use of the antibody of any one of E1-E23 in the manufacture of a medicament for use in the treatment of cancer.

E41. A pharmaceutical composition for use in the treatment of cancer, comprising the antibody of any one of E1-E23.

E42. Use of the antibody of any one of E1-E23 to treat cancer.

E43. The antibody, polynucleotide(s), method, pharmaceutical composition, or use of any of E1-E42, wherein the cancer is bladder cancer, breast cancer, clear cell kidney cancer, head/neck squamous cell carcinoma [squamous cell carcinoma of the head and neck (SCCHN)], lung squamous cell carcinoma, lung adenocarcinoma, malignant melanoma, non-small-cell lung cancer (NSCLC), ovarian cancer, pancreatic cancer, prostate cancer, renal cell carcinoma (RCC), small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome 35 (MDS), non-Hodgkin's lymphoma (NHL), small lymphocytic lymphoma (SLL), endometrial cancer, B-cell acute lymphoblastic leukemia, colorectal cancer (CRC), glioblastoma, uterine cancer, cervical cancer, penile cancer, gastric cancer (GC) or non-melanoma skin cancer.

E44. The antibody, polynucleotide, method, pharmaceutical composition, or use of E43, wherein the cancer was previously treated with a PD1 or PDL1 [PD(L)1] inhibitor.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd, edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989): Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of anti-LTBR antibody) means that the parameter may vary by as much as 10% below or above the stated numerical value for that parameter. For example, a dose of about 5 mg means 5%±10%, i.e. it may vary between 4.5 mg and 5.5 mg.

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g. monospecific, bispecific), and includes portions of intact antibodies that retain the ability to bind to a given 20 antigen (e.g. an “antigen-binding fragment”), and any other modified configuration of an immunoglobulin molecule that comprises an antigen binding site.

An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The extended definition is the combination of the Kabat and Chothia definitions. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford. UK; Oxford Molecular. Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions.

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL)

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include Clq binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in 20 contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

An “antigen” refers to the molecular entity used for immunization of an immunocompetent vertebrate to produce the antibody that recognizes the antigen or to screen an expression library (e.g., phage, yeast or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition or alternatively, during the discovery process, the generation and characterization of antibodies may elucidate information about desirable epitopes. From this information, it is then possible to competitively screen antibodies for binding to the same epitope.

In addition, the epitope to which an antibody binds can be determined in a systematic screening by using overlapping peptides derived from the antigen and determining binding by the antibody. According to the gene fragment expression assays, the open reading frame encoding the antigen can be fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis.

Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, or necessary for epitope binding.

At its most detailed level, the epitope for the interaction between the antigen and the antibody can be defined by the spatial coordinates defining the atomic contacts present in the antigen-antibody interaction, as well as information about their relative contributions to the binding thermodynamics. At a less detailed level, the epitope can be characterized by the spatial coordinates defining the atomic contacts between the antigen and antibody. At a further less detailed level the epitope can be characterized by the amino acid residues that it comprises as defined by a specific criterion, e.g., by distance between atoms (e.g., heavy, i.e., non-hydrogen atoms) in the antibody and the antigen. At a further less detailed level the epitope can be characterized through function, e.g., by competition binding with other antibodies. The epitope can also be defined more generically as comprising amino acid residues for which substitution by another amino acid will alter the characteristics of the interaction between the antibody and antigen (e.g. using alanine scanning).

From the fact that descriptions and definitions of epitopes, dependent on the epitope mapping method used, are obtained at different levels of detail, it follows that comparison of epitopes for different antibodies on the same antigen can similarly be conducted at different levels of detail.

Epitopes described at the amino acid level, e.g., determined from an X-ray crystallography, Nuclear Magnetic Resonance (NMR) spectroscopy, hydrogen/deuterium exchange Mass Spectrometry (H/D-MS), are said to be identical if they contain the same set of amino acid residues. Epitopes are said to overlap if at least one amino acid is shared by the epitopes. Epitopes are said to be separate (unique) if no amino acid residue is shared by the epitopes.

Yet another method which can be used to characterize an antibody is to use competition assays with other antibodies known to bind to the same antigen, to determine if an antibody of interest binds to the same epitope as other antibodies. Competition assays are well known to those of skill in the art. Epitopes characterized by competition binding are said to be overlapping if the binding of the corresponding antibodies are mutually exclusive, i.e., binding of one antibody excludes simultaneous or consecutive binding of the other antibody. The epitopes are said to be separate (unique) if the antigen is able to accommodate binding of both corresponding antibodies simultaneously.

Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds.

The term “binding affinity” refers to the strength of the sum total of noncovalent interactions between a single binding site of a molecule (e.g., an antibody) and its binding partner (e.g., an antigen). Unless indicated otherwise, as used herein. “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (k_{off})” or “k_{d}” to the association rate, or “on-rate (kₒₙ)” or “kₐ”. Thus, K_{D} equals k_{off}/kₒₙ (or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (Octet QK® system, ForteBio). Alternatively, or in addition, a KinExA (Kinetic Exclusion Assay) assay, available from Sapidyne Instruments (Boise, ID) can also be used.

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the antibody can bind multiple antigen molecules

A “bispecific antibody” refers to a molecule that has binding specificity for at least two different epitopes. In some embodiments, bispecific antibodies can bind simultaneously two different antigens. In other embodiments, the two different epitopes may reside on the same antigen.

A “tetravalent” antibody refers to an antibody that comprises four antigen binding sites per molecule. A tetravalent antibody can be monospecific (i.e. such that all four binding sites of the tetravalent antibody specifically recognize the same epitope on an antigen), bispecific, trispecific, or tetraspecific. Unless otherwise noted, tetravalent antibodies provided herein are monospecific.

A “bivalent” antibody refers to an antibody that comprises two antigen binding sites per molecule. A bivalent antibody can be monospecific (i.e. such that both binding sites of the bivalent antibody specifically recognize the same epitope on an antigen) or bispecific. Unless otherwise noted, bivalent antibodies provided herein are monospecific.

The term “half maximal effective concentration (EC₅₀)” refers to the concentration of a therapeutic agent which causes a response halfway between the baseline and maximum after a specified exposure time. The therapeutic agent may cause inhibition or stimulation. The EC₅₀ value is commonly used, and is used herein, as a measure of potency.

The term “half maximal inhibitory concentration (IC₅₀)” refers to the concentration of a therapeutic agent that is needed to inhibit a given biological process, function or component by 50%. An IC₅₀ is a measure of potency of a substance (e.g., a therapeutic agent) in inhibiting a specific biological process, function or component.

An “agonist” refers to a substance which promotes (i.e., induces, causes, enhances, or increases) the biological activity or effect of another molecule. The term agonist encompasses substances (such as an antibody) which bind to a molecule to promote the activity of that molecule.

An “antagonist” refers to a substance that prevents, blocks, inhibits, neutralizes, or reduces a biological activity or effect of another molecule, such as a receptor. The term antagonist encompasses substances (such as an antibody) which bind to a molecule to prevent or reduce the activity of that molecule.

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

Standard competition assays may be used to determine whether two antibodies compete with each other. One suitable assay for antibody competition involves the use of the Biacore technology, which can measure the extent of interactions using surface plasmon resonance (SPR) technology, typically using a biosensor system (such as a BIACORE system). For example, SPR can be used in an in vitro competitive binding inhibition assay to determine the ability of one antibody to inhibit the binding of a second antibody. Another assay for measuring antibody competition uses an ELISA-based approach.

An “Fc receptor” (FcR) refers to a receptor that binds to the Fc domain of an antibody. In some embodiments, an FcR is a native human FcR. In some embodiments, an FcR is one which binds an IgG antibody (a gamma receptor; (FcgR)) and includes receptors of the FcgRI, FcgRII, and FcgRIII subclasses, including allelic variants and alternatively spliced forms of those receptors. FcgRII receptors include FcgRIIA (an “activating receptor”) and FcgRIIB (an “inhibiting receptor”), which have similar amino acid sequences that differ primarily in the cytoplasmic domains thereof. Activating receptor FcgRIIA contains an immunoreceptor tyrosine-based activation motif (ITAM) in its cytoplasmic domain. Inhibiting receptor FcgRIIB contains an immunoreceptortyrosine-based inhibition motif (ITIM) in its cytoplasmic domain, (see, e.g., Daeron, Annu. Rev. Immunol. 1997; 15:203-234). FcRs are reviewed, for example, in Ravetch and Kinet, Annu. Rev. Immunol 1991: 9:457-92; Capel et al., Immunomethods 1994; 4:25-34; and de Haas et al., J. Lab. Clin. Med. 1995; 126:330-41. Other FcRs, including those to be identified in the future, are encompassed by the term “Fc receptor” herein. The term “Fc receptor” also includes the neonatal receptor, FcRn, which is responsible for the transfer of maternal IgGs to the fetus (Guyer et al., J. Immunol. 1976; 117:587 and Kim et al., J. Immunol. 1994; 24:249) and regulation of homeostasis of immunoglobulins. Methods of measuring binding to FcRn are known (see, e.g., Ghetie and Ward., Immunol. Today 1997; 18(12):592-598; Ghetie et al., Nature Biotechnology, 1997; 15(7):637-640: Hinton et al., J. Biol. Chem. 2004; 279(8):6213-6216; WO 2004/92219).

A “fragment” or “portion” of an antibody or polypeptide may be made by truncation, e.g., by removal of one or more amino acids from the amino terminal end, the carboxy terminal end or both ends of a polypeptide. One, 2, 3, 4, 5, 6, 7, 8, 9, 10, up to 20, up to 30 up to 40, up to 50, up to 60, up to 70, up to 80 up to 100 or more amino acids may be removed from the amino terminal end, the carboxy terminal end or both ends of the polypeptide to produce a fragment or portion. A fragment or portion may be made by one or more deletions of amino acids from the polypeptide. A fragment or portion may be made by one or more deletions of amino acids from the polypeptide as well as removal of one or more amino acids from the amino terminal end, the carboxy terminal end or both ends of a polypeptide.

An “effector cell” refers to a leukocyte which express one or more FcRs and performs effector functions. In certain embodiments, effector cells express at least FcgRIII and perform ADCC effector function(s). Examples of leukocytes which mediate ADCC include peripheral blood mononuclear cells (PBMC), natural killer (NK) cells, monocytes, macrophages, cytotoxic T cells, and neutrophils. Effector cells may be isolated from a native source, e.g., from blood.

The term “antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcgRIII only, whereas monocytes express FcgRI, FcgRII, and FcgRIII. To assess ADCC activity of a molecule of interest, an in vitro ADCC assay, such as that described in U.S. Pat. Nos. 5,500,362, 5,821,337 or 6,737,056, may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA) 1998; 95:652-656. Additional antibodies with altered Fc domain amino acid sequences and increased or decreased ADCC activity are described, e.g., in U.S. Pat. Nos. 7,923,538, and 7,994,290.

The term “altered” FcR binding affinity or ADCC activity refers to an antibody which has either enhanced or diminished activity for one or more of FcR binding activity or ADCC activity compared to a parent antibody, wherein the antibody and the parent antibody differ in at least one structural aspect. An antibody that “displays increased binding” to an FcR binds at least one FcR with better affinity than the parent antibody. An antibody that “displays decreased binding” to an FcR, binds at least one FcR with lower affinity than a parent antibody. Such antibodies that display decreased binding to an FcR may possess little or no appreciable binding to an FcR, e.g., 0-20 percent binding to the FcR compared to a native sequence IgG Fc domain.

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

A “vector” refers to a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc.). Vectors may include DNA or RNA. An “expression vector” as used herein refers to a vector that includes at least one polypeptide-encoding gene, at least one regulatory element (e.g. promoter sequence, poly(A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers and terminator). For translation, one or more translational controlling elements may also be included such as ribosome binding sites, translation initiation sites, and stop codons.

An “isolated” molecule (e.g. antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art.

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “polynucleotide” or “nucleic acid,” (used interchangeably herein) refers to a chain of nucleotides of any length, and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside.

A “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine. Exemplary potential conservative substitutions include the following amino acid pairs that may be substituted: Ala/Val; Arg/Lys; Asn/Gln; Asp/Glu; Cys/Ser; Gln/Asn; Glu/Asp; Gly/Ala; His/Arg; Ile/Leu; Met/Leu; Phe/Tyr; Pro/Ala; Ser/Thr; Trp/Tyr; Val/Leu.

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g. algorithms), which are well known in the art.

Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, sequences can be aligned using the methods and computer programs, including BLAST, available over the world wide web at ncbi.nlm.nih.gov/BLAST/. Other alignment programs include MegAlign® program in the Lasergene® suite of bioinformatics software (DNASTARO, Inc., Madison, WI). Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in 35 sequence alignments. See Meth. Mal. Biol. 70: 173-187 (1997). Also, the GAP program using the Needleman and Wunsch alignment method can be utilized to align sequences. See J. Mal. Biol. 48: 443-453 (1970).

Also, of interest is the BestFit program using the local homology algorithm of Smith and Waterman (1981, Advances in Applied Mathematics 2: 482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, WI, USA.

Another program of interest is the FastDB algorithm. FastDB is described in Current Methods in Sequence Comparison and Analysis, Macromolecule Sequencing and Synthesis. Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

The term ‘excipient’ refers to any material which, which combined with an active ingredient of interest (e.g. antibody), allow the active ingredient to retain biological activity. The choice of excipient will to a large extent depend on factors such as the mode of administration, the effect of the excipient on solubility and stability, and the nature of the dosage form. As used herein, “excipient”” includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder, or condition is cancer.

The terms “prevent” or “prevention” refer to preventing a disease, condition or disorder in an individual that may be predisposed to the disease, condition or disorder but does not yet experience or display the pathology or symptomatology of the disease. In some embodiments, prevention is assessed on a population basis such that an agent is considered to “prevent” a particular disease, disorder or condition if a statistically significant decrease in the development, frequency or intensity of the disease, disorder or condition is observed in a population susceptible to the disease, disorder or condition. Prevention may be considered complete when onset of disease, disorder or condition has been delayed for a predefined period of time.

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development. In some embodiments, a subject is a patient with cancer.

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following:
(1) preventing the disease: for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and
(3) ameliorating the disease; for example, ameliorating a disease, condition or disorder in an individual that is experiencing or displaying the pathology or symptomatology of the disease, condition or disorder (i.e., reversing the pathology or symptomatology).

### Antibodies to LTBR

Provided herein are antibodies that bind to human lymphotoxin beta receptor (LTBR) (also known as Tumor Necrosis Factor Receptor superfamily member 3 (TNFR3).

As used herein, the term “LTBR” includes variants, isoforms, homologs, orthologs and paralogs of LTBR. In some embodiments, an antibody disclosed herein cross-reacts with LTBR from species other than human, such as LTBR of cynomolgus monkey, as well as different forms of LTBR. In some embodiments, an antibody may be completely specific for human LTBR and may not exhibit species cross-reactivity (e.g., does not bind mouse LTBR) or other types of cross-reactivity (e.g., does not bind other receptors in the tumor necrosis factor receptor family). As used herein the term LTBR refers to naturally occurring human LTBR unless contextually dictated otherwise. Therefore, a “LTBR antibody” “anti-LTBR antibody” or other similar designation means any antibody (as defined herein) that binds or reacts with LTBR, an isoform, fragment or derivative thereof.

The full length, mature form of human LTBR, as represented by UniProtKB/Swiss-Prot accession number p36941-1 is herein provided as SEQ ID NO: 15:

(SEQ ID NO: 15)
MLLPWATSAPGLAWGPLVLGLFGLLAASQPQAVPPYASENQTCRDQEKEY
YEPQHRICCSRCPPGTYVSAKCSRIRDTVCATCAENSYNEHWNYLTICQL
CRPCDPVMGLEEIAPCTSKRKTQCRCQPGMFCAAWALECTHCELLSDCPP
GTEAELKDEVGKGNNHCVPCKAGHFQNTSSPSARCQPHTRCENQGLVEAA
PGTAQSDTTCKNPLEPLPPEMSGTMLMLAVLLPLAFFLLLATVFSCIWKS
HPSLCRKLGSLLKRRPQGEGPNPVAGSWEPPKAHPYFPDLVQPLLPISGD
VSPVSTGLPAAPVLEAGVPQQQSPLDLTREPQLEPGEQSQVAHGTNGIHV
TGGSMTITGNIYIYNGPVLGGPPGPGDLPATPEPPYPIPEEGDPGPPGLS
TPHQEDGKAWHLAETEHCGATPSNRGPRNQFITHD

The full length, mature form of mouse LTBR, as represented by UniProtKB/Swiss-Prot accession number P50284 is herein provided as SEQ ID NO: 16:

(SEQ ID NO: 16)
MRLPRASSPCGLAWGPLLLGLSGLLVASQPQLVPPYRIENQTCWDQDKEY
YEPMHDVCCSRCPPGEFVFAVCSRSQDTVCKTCPHNSYNEHWNHLSTCQL
CRPCDIVLGFEEVAPCTSDRKAECRCQPGMSCVYLDNECVHCEEERLVLC
QPGTEAEVTDEIMDTDVNCVPCKPGHFQNTSSPRARCQPHTRCEIQGLVE
AAPGTSYSDTICKNPPEPGAMLLLAILLSLVLFLLFTTVLACAWMRHPSL
CRKLGTLLKRHPEGEESPPCPAPRADPHFPDLAEPLLPMSGDLSPSPAGP
PTAPSLEEVVLQQQSPLVQARELEAEPGEHGQVAHGANGIHVTGGSVTVT
GNIYIYNGPVLGGTRGPGDPPAPPEPPYPTPEEGAPGPSELSTPYQEDGK
AWHLAETETLGCQDL

The full length, mature form of cynomolgus LTBR, as represented by UniProtKB/Swiss-Prot accession number A0A2K5VGQ6 is herein provided as SEQ ID NO: 17:

(SEQ ID NO: 17)
MRLPWATSAPGLAWGPLVLGLFGLLAASQPQVVRKGPVPPYGSENQTCRD
QEKEYYEPRHRICCSRCPPGTYVSAKCSRSRDTVCATCAENSYNEHWNYL
TICQLCRPCDPVMGLEEIAPCTSKRKTQCRCQPGMFCAAWALECTHCELL
SDCPPGTEAELKDEVGKGNNHCVPCKAGHFQNTSSPSARCQPHTRCEDQG
LVEAAPGTAQSDTTCRNPSESLPPEMSGTMLMLAILLPLAFFLLLATIFA
CIWKSHPSLCRKLGSLLKRHPQGEGPNPVAAGRDPPKANPQYPDLVEPLL
PISGDVSPVSTGLPTALVSEEGVPQQQSPLDLTTEPQLEPGEQNQVAHGT
NGIHVTGGSMTITGNIYIYNGPVLGGPPGPGDLPATPDPPYPIPEEGDPG
PPGLSTPHQEDGKAWHLAETEHCGATPSNRGPRSQFITYD

LTBR is broadly expressed on parenchymal and stromal cells, epithelial and endothelial cells, and myeloid cells. It has pleiotropic immune stimulatory functions including dendritic cell stimulation, T cell priming and activation, immune cell recruitment, and plays a critical role in the formation and structure of secondary lymphoid organs and tertiary lymphoid structures (TLS).

The natural ligands of LTBR are LIGHT (which assembles as a trimer) and the LTalpha1beta2 (LTa1B2) heterotrimer [containing lymphotoxin alpha (LTa) and lymphotoxin beta (LTB)]. Binding of LIGHT and LTa1B2 to LTBR causes clustering of LTBR molecules, and initiates activation of various downstream LTBR signaling pathways. For example, activation of LTBR results in activation of Nuclear Factor kappa-light-chain-enhancer of activated B cells (NFkB) and NFkB signaling, and increased expression of genes such as IL12, type I IFN, CXCL9, CXCL10, CXCL13, CCL19, and CCL21. Engagement of LTBR also results in upregulation of adhesion receptors and chemokines in a cell context-dependent manner to orchestrate lymphoid neogenesis and dendritic cell homeostasis.

Without wishing to be bound by any particular theory, binding of an antibody provided herein to LTBR results in clustering of multiple LTBR molecules on the cell surface, thereby promoting LTBR-mediated signaling. Different formats of anti-LTBR antibody can promote LTBR clustering.

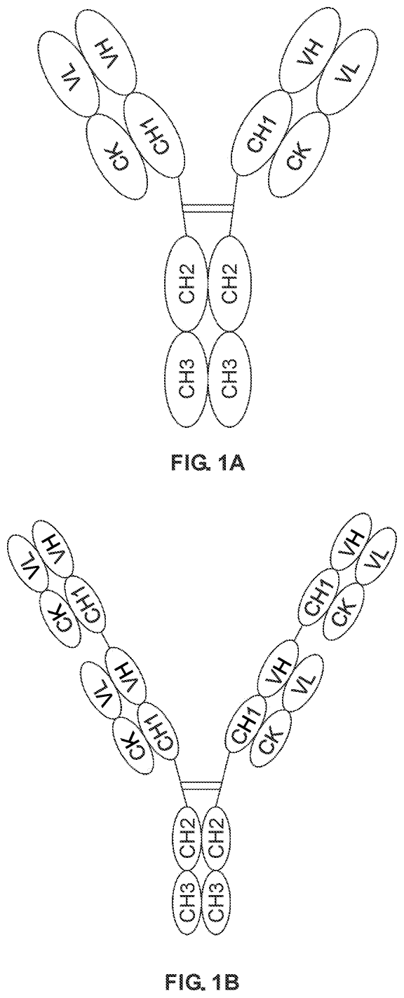

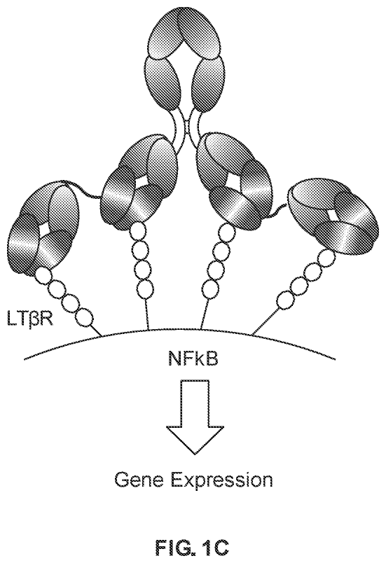

For example, in some embodiments, a bivalent anti-LTBR antibody can promote LTBR clustering. A bivalent anti-LTBR antibody can promote LTBR clustering through multiple different mechanisms. For example, binding of each antigen binding site of a bivalent anti-LTBR antibody to a separate LTBR molecule and lead to direct clustering of two LTBR molecules. If a bivalent anti-LTBR antibody contains an Fc domain that binds to an Fc receptor (e.g. as present on various types of immune cells), binding of an immune cell to the Fc domain of two separate bivalent anti-LTBR antibodies can result in the clustering of four LTBR molecules (e.g. two LTBR molecules bound by each of the two bivalent antibodies). In some embodiments, provided herein is a bivalent anti-LTBR antibody having the structure as shown in FIG. 1A.

In some embodiments, a tetravalent anti-LTBR antibody can promote LTBR clustering. A tetravalent anti-LTBR antibody can promote LTBR clustering through multiple different mechanisms. For example, binding of each antigen binding site of a tetravalent anti-LTBR antibody to a separate LTBR molecule and lead to direct clustering of four LTBR molecules. Then, if a tetravalent anti-LTBR antibody contains an Fc domain that binds to an Fc receptor, binding of an immune cell to the Fc domain of two separate tetravalent anti-LTBR antibodies can result in the clustering of up to 8 LTBR molecules (e.g. four LTBR molecules bound by each of the two tetravalent antibodies). Even if a tetravalent anti-LTBR antibody lacks an Fc domain or lacks an Fc domain that binds to an Fc receptor, it can still lead to direct clustering of four LTBR molecules due to each antigen binding site of the tetravalent antibody binding to a separate LTBR molecule.

In some embodiments, an anti-LTBR antibody of the disclosure encompasses an antibody that one or both of i) competes for binding to human LTBR with or ii) binds the same epitope as, an antibody comprising the amino acid sequence of a heavy chain variable region set forth as SEQ ID NO: 10 and the amino acid sequence of a light chain variable region set forth as SEQ ID NO: 11.

Anti-LTBR antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-LTBR antibody is a monoclonal antibody. In some embodiments, an anti-LTBR antibody is a human or humanized antibody. In some embodiments, an anti-LTBR antibody is a chimeric antibody.

In some embodiments, the invention provides an antibody having a light chain variable region (VL) sequence and a heavy chain variable region (VH) sequence as found in Table 1, or variants thereof. In Table 1, the underlined sequences are CDR sequences (Kabat definition) and the sequences in bold are CDR sequences (Chothia definition).

TABLE 1

| ID | Sequence |
|---|---|
| 1E01 | QVQLVQSGAEVKKPGSSVKVSCKASGGTFSSYFWSWVRQAPGQG |
| VH | LEWMGRFYSGGSANYNPSLKERVTITADESTSTAYMELSSLRSE |
| | DTAVYYCARERRGYSGGFEIWGQGTLVTVSS (SEQ ID NO: |
| | 10) |
| 1E01 | DIVMTQSPLSLPVTPGEPASISCRSSQSLLHSNSYNYLDWYLQK |
| VL | PGQSPQLLIYLGSYRASGVPDRFSGSGSGTDFTLKISRVEAEDV |
| | GVYYCMQPLQTPFTFGPGTKVDIK (SEQ ID NO: 11) |

The invention also provides CDR portions of antibodies to LTBR. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the antibody comprises three CDRs of the heavy chain variable region shown in Table 1. In some embodiments, the antibody comprises three CDRs of the light chain variable region shown in Table 1. In some embodiments, the antibody comprises three CDRs of the heavy chain variable region shown in Table 1, and three CDRs of the light chain variable region shown in Table 1.

Table 2 provides examples of CDR sequences of anti-LTBR antibodies provided herein.

TABLE 2
Anti-LTBR antibodies (mAbs) and their
antigen-binding CDR sequences

| mAb | CDR1 | CDR2 | CDR3 |
|---|---|---|---|
| 1E01 | GGTFSSY (SEQ ID | YSGGS (SEQ ID NO: | ERRGYSGGF |
| VH | NO: 1)(Chothia) | 4)(Chothia) | EI (SEQ |
| | SYFWS (SEQ ID | RFYSGGSANYNPSLKE | ID NO: 6) |
| | NO: 2) (Kabat) | (SEQ ID NO: 5) | |
| | GGTFSSYFWS (SEQ | (Kabat and | |
| | ID NO: 3) | Extended) | |
| | (Extended) | | |
| 1E01 | RSSQSLLHSNSYNYL | LGSYRAS (SEQ ID | MQPLQTPFT |
| VL | D (SEQ ID NO: | NO: 8) | (SEQ ID |
| | 7) | | NO: 9) |

In some embodiments, the antibody comprises three light chain CDRs and three heavy chain CDRs from Table 2.

In some embodiments, the antibody comprises one or both of D) the full-length heavy chain, with or without the C-terminal lysine, or ii) the full-length light chain of anti-LTBR antibody 1E01 in bivalent, trivalent, or tetravalent format. The amino acid sequences of the full-length heavy chain and light chain for antibodies 1E01 in bivalent and tetravalent formats are shown below in Table 3. The different 1E01 formats (e.g. bivalent and tetravalent) contain the same VH and VL sequences (SEQ ID NO: 10 and SEQ ID NO: 11, respectively) (in the tetravalent heavy chain, the VH sequence is present twice).

TABLE 3
Heavy chain and light chain sequences for
mAbs of the invention

| ID | Sequence |
|---|---|
| 1E01 | QVQLVQSGAEVKKPGSSVKVSCKASGGTFSSYFWSWVRQAPGQGLEWMGRFY |
| Bivalent | SGGSANYNPSLKERVTITADESTSTAYMELSSLRSEDTAVYYCARERRGYSG |
| Heavy | GFEIWGQGTLVTVSSASTKGPSVFPLAPSSKSTSGGTAALGCLVKDYFPEPV |
| chain | TVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEMTKNQ |
| | VSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 14) |
| 1E01 | QVQLVQSGAEVKKPGSSVKVSCKASGGTFSSYFWSWVRQAPGQGLEWMGRFY |
| Tetra- | SGGSANYNPSLKERVTITADESTSTAYMELSSLRSEDTAVYYCARERRGYSG |
| valent | GFEIWGQGTLVTVSSASTKGPSVFPLAPSSKSTSGGTAALGCLVKDYFPEPV |
| Heavy | TVSWNSGALTSGVHTFPAVLQSSGLYSLSSVVTVPSSSLGTQTYICNVNHKP |
| chain | SNTKVDKKVEPKSCGGGGSQVQLVQSGAEVKKPGSSVKVSCKASGGTFSSYF |
| The linker | WSWVRQAPGQGLEWMGRFYSGGSANYNPSLKERVTITADESTSTAYMELSSL |
| is in bold | RSEDTAVYYCARERRGYSGGFEIWGQGTLVTVSSASTKGPSVFPLAPSSKST |
| text | SGGTAALGCLVKDYFPEPVTVSWNSGALTSGVHTFPAVLQSSGLYSLSSVVT |
| Mutations | VPSSSLGTQTYICNVNHKPSNTKVDKKVEPKSCDKTHTCPPCPAPEAAGAPS |
| L234A, | VFLFPPKPKDTLMISRTPEVTCVVVDVSHEDPEVKFNWYVDGVEVHNAKTKP |
| L235A, | REEQYNSTYRVVSVLTVLHQDWLNGKEYKCKVSNKALPAPIEKTISKAKGQP |
| and | REPQVYTLPPSREEMTKNQVSLTCLVKGFYPSDIAVEWESNGQPENNYKTTP |
| G237A | PVLDSDGSFFLYSKLTVDKSRWQQGNVFSCSVMHEALHNHYTQKSLSLSPGK |
| are | (SEQ ID NO: 12) |
| underlined | |
| 1E01 | DIVMTQSPLSLPVTPGEPASISCRSSQSLLHSNSYNYLDWYLQKPGQSPQLL |
| Bivalent | IYLGSYRASGVPDRFSGSGSGTDFTLKISRVEAEDVGVYYCMQPLQTPFTFG |
| and | PGTKVDIKRTVAAPSVFIFPPSDEQLKSGTASVVCLLNNFYPREAKVQWKVD |
| Tetra- | NALQSGNSQESVTEQDSKDSTYSLSSTLTLSKADYEKHKVYACEVTHQGLSS |
| valent | PVTKSFNRGEC (SEQ ID NO: 13) |
| Light | |
| chain | |

In some embodiments, provided herein is a tetravalent anti-LTBR antibody having the structure as shown in FIG. 1B.

In some embodiments, provided herein is a tetravalent anti-LTBR antibody having the structure as shown in FIG. 1B and one or more of the following features: The tetravalent antibody has two extended heavy chains, and four light chains. Each extended heavy chain has, in N-terminus to C-terminus order, a VH, CH1, VH, CH1, CH2, CH3 domain, and the CH1 and CH2 domains are separated by a hinge region. The first CH1 is separated from the second VH by a short linker polypeptide, having the amino acid sequence EPKSCGGGGS (SEQ ID NO: 18). There are four antigen binding sites in this molecule (the four VHNL pairs). Each heavy chain of the antibody comprises the amino acid sequence of SEQ ID NO: 12 and each light chain comprises the amino sequence of SEQ ID NO: 13. The heavy chain of SEQ ID NO: 12 also contains mutations in the IgG1 CH2 domain (L234A, L235A, and G237A: EU numbering) to reduce IgG1-Fc domain effector function.

In some embodiments, a tetravalent anti-LTBR antibody provided herein is monospecific. For example, a tetravalent anti-LTBR antibody comprising a heavy chain comprising the amino acid sequence of SEQ ID NO: 12 and a light chain comprising the amino sequence of SEQ ID NO: 13 is monospecific.

FIG. 1C depicts a schematic of a tetravalent anti-LTBR antibody as shown in FIG. 1B binding to 4 separate LTBR molecules, and thereby promoting LTBR clustering and LTBR-mediated signaling. As further shown in FIG. 1C, clustering of LTBR results in signaling such as NFkB signaling, and downstream signaling of the NFkB pathways, such as increased expression of genes encoding IL12, type I IFN, CXCL9, CXCL10, CXCL13, CCL19, and CCL21. Thus, LTBR clustering can be identified by resulting downstream cytokine production.

In some embodiments, provided herein is a bivalent anti-LTBR antibody having the structure as shown in FIG. 1A.

In some embodiments, provided herein is a bivalent anti-LTBR antibody having the structure as shown in FIG. 1A and one or more of the following features: The bivalent antibody has two heavy chains and two light chains. Each heavy chain has, in N-terminus to C-terminus order, a VH, CH1, CH2, and CH3 domain, and the CH1 and CH2 domains are separated by a hinge region. There are two antigen binding sites in this molecule (the two VHNL pairs). The heavy chain of the antibody comprises the amino acid sequence of SEQ ID NO: 14 and the light chain comprises the amino sequence of SEQ ID NO: 13.

In certain embodiments, an antibody described herein comprises an Fc domain. The Fc domain can be derived from IgA (e.g., IgA₁ or IgA₂), IgG, IgE, or IgG (e.g., IgG₁, IgG₂, IgG₃, or IgG₄). In some embodiments, an anti-LTBR antibody is an IgG1 antibody.

The invention encompasses modifications to the variable regions shown in Table 1, the CDRs shown in Table 2, and heavy chain and light chain sequences shown in Table 3. For example, the invention includes antibodies comprising functionally equivalent variable regions and CDRs which do not significantly affect their properties as well as variants which have enhanced or decreased activity or affinity. For example, the amino acid sequence may be mutated to obtain an antibody with the desired binding affinity to LTBR. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

A modification or mutation may also be made in a framework region or constant region to increase the half-life of an antibody provided herein. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

For example, in some embodiments, the constant region of an antibody provided herein is modified to have decreased binding affinity to a human Fc gamma receptor. Such antibodies may have, for example, one or more of the mutations L234A, L235A, and G237A in the IgG1 CH2 domain to reduce effector function (EU numbering).

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given 30 glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyttransferase III (GnTIII), a glycosyltransferase catalyzing formation of bisecting GlcNAc, was reported to have improved ADCC activity (Umana et al., 1999, Nature Biotech. 17:176-180).

In some embodiments, the disclosure provides anti-LTBR antibodies containing variations of the variable regions shown in Table 1, the CDRs shown in Table 2, or heavy chain and light chain sequences shown in Table 3, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in Table 1, 2, or 3. These amounts are not meant to be limiting and increments between the recited percentages are specifically envisioned as part of the disclosure.

In some embodiments provided herein is an anti-LTBR antibody that comprises a VH and a VL, wherein the antibody VH has an amino acid sequence encoded by the nucleic acid sequence of the insert of the plasmid deposited with the ATCC having ATCC Accession No. PTA-127515 and the antibody VL has an amino acid sequence encoded by the nucleic acid sequence of the insert of the plasmid deposited with the ATCC having ATCC Accession No. PTA-127516. In some embodiments, the anti-LTBR antibody is tetravalent.

The invention also encompasses fusion proteins comprising one or more components of the antibodies disclosed herein. In some embodiments, a fusion protein may be made that comprises all or a portion of an anti-LTBR antibody of the invention linked to another polypeptide. In another embodiment, only the variable domains of the anti-LTBR antibody are linked to the polypeptide. In another embodiment, the VH domain of an anti-LTBR antibody is linked to a first polypeptide, while the VL domain of an anti-LTBR antibody is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

### Biological Activity of Anti-LTBR Antibodies

In addition to binding an epitope on LTBR, an antibody of the disclosure can mediate a biological activity. That is, the disclosure includes an isolated antibody that specifically binds LTBR and mediates at least one detectable activity selected from the following:
(i) binds specifically to human LTBR;
(ii) binds specifically to cynomolgus monkey LTBR;
(iii) promotes the clustering of LTBR on the cell membrane;
(iv) promotes LTBR-mediated NFkB activation or NFkB downstream signaling
(v) induces Cxcl10 secretion
(vi) induces IL-12 secretion
(vii) inhibits tumor growth.

Without wishing to be bound by any particular theory, binding of an antibody provided herein to LTBR results in clustering of multiple LTBR molecules on the cell surface, thereby promoting LTBR-mediated signaling. In addition, without being bound by theory, agonizing LTBR by an antibody provided herein enhances anti-tumor immunity via dendritic cell and T cell priming, immune cell recruitment, and TLS induction and maturation, and can result in clinical activity in PD(L)1 inhibitor-resistant tumors. For example, in one embodiment, an anti-LTBR antibody provided herein may bind to LTBR present on dendritic cells, resulting in enhanced priming of T effector cells by dendritic cells. In another embodiment, an anti-LTBR antibody provided herein may bind to LTBR present on stromal cells, resulting in recruitment of immune cells into a tumor. In another embodiment, an anti-LTBR antibody provided herein may bind to LTBR present on endothelial cells, resulting in the generation of structural conduits for concentrating immune cells in a tumor and the induction of lymphoid structures.

In one aspect, the clustering of LTBR on the cell membrane by an anti-LTBR antibody may be assessed by measuring LTBR receptor occupancy on cells, such as peripheral blood monocytes.

In some embodiments, binding of an antibody herein to LTBR promotes NFkB activation and signaling. In one aspect, NFkB activation and signaling may be assessed by measuring expression of markers downstream in the NFkB pathway (e.g. ICAM-1) in cells, such as peripheral blood monocytes.

In some embodiments, binding of an antibody herein to LTBR promotes one or both of the formation and maturation of tertiary lymphoid structures (TLS). The structure and role of TLS in cancer are described, for example, in Schumacher et al, Science, 375, 39 (2022).

In one aspect, the increase in formation of TLS by an anti-LTBR antibody may be assessed by histology to examine tumors for the formation of TLS [e.g. using immunohistochemistry (IHC) or immunofluorescence (IF) based analysis]. Exemplary markers to use for detecting and quantifying TLS in tumor biopsies can include CD3, CD8, CD20, CD21, CD23, DC-LAMP, and PNAd. Tumor samples may be obtained, for example, by core needle biopsy.

In another aspect, the increase in formation of TLS by an anti-LTBR antibody may be assessed by examining gene expression in tumors by RNA sequences to assess for a gene expression signature indicative of TLS induction. Gene expression signatures indicative of TLS induction are described, for example, in Coppola et al. Am J. Pathol, 2011, 179: 37 and Messina et al. Scientific Report, 2012; 2: 765. Gene expression that may be assessed include, for example, genes encoding IL12, type I IFN, CXCL9, CXCL10, CXCL13, CCL19, and CCL21.

In another aspect, the increase in formation of TLS by an anti-LTBR antibody may be assessed by examining cytokine (e.g. including chemokines) expression in tumors and peripheral blood to assess for cytokine expression indicative of TLS induction. Cytokines that may be assessed in include, for example, IL12, type I IFN, CXCL9, CXCL10, CXCL13, CCL19, and CCL21.

In another aspect, the increase in formation of TLS by an anti-LTBR antibody may be assessed by examining peripheral blood to assess for LTBR and TLS-related immune cell subtypes in the blood.

In some embodiments, binding of an antibody herein to LTBR promotes the inhibition of tumor growth in PD(L)1 inhibitor therapy resistant cancer cells [i.e. cancer cells that are resistant to treatment with PD-1 and/or PD-L1 inhibitors].

In some embodiments, binding of an antibody herein to LTBR promotes the infiltration of CD8 positive (CD8+) T cells in the tumor microenvironment.

In some embodiments, binding of an antibody herein to LTBR promotes the production of CXCL13. CXCL13 is a chemokine produced by immune cells such as T cells, and is involved multiple inflammatory and immune responses, such as the development of tertiary lymphoid structures (TLS).

### Polynucleotides Encoding Anti-LTBR Antibodies, and Methods of Manufacture

In some embodiments, provided herein are polynucleotides encoding any of the antibodies provided herein, including antibody portions and modified antibodies. Also included are methods of making any of the antibodies and polynucleotides described herein. Polynucleotides can be made and the proteins expressed by procedures known in the art.

An anti-LTBR antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide or polynucleotide(s) comprising a sequence encoding one or both of the heavy chain or the light chain variable regions of an anti-LTBR antibody provided herein. The polynucleotide(s) encoding the antibody of interest may be maintained in a vector in a host cell and the host cell can then be expanded and frozen for future use. Vectors (including expression vectors) and host cells are further described herein.

In some embodiments, the disclosure provides polynucleotides encoding the amino acid sequences of any one of the 1E01 bivalent or tetravalent anti-LTBR antibodies.

In some embodiments, the disclosure provides polynucleotides encoding one or more anti-LTBR antibody heavy chain polypeptides comprising an amino acid sequence selected from the group consisting of: SEQ ID NOs: 12 or 14. In some embodiments, a polynucleotide comprising the nucleotide sequence of SEQ ID NO: 19 encodes a heavy chain polypeptide comprising the amino acid sequence of SEQ ID NO: 12.

### SEQ ID NO: 19 is:

(SEQ ID NO: 19)
CAAGTGCAGCTTGTGCAGAGCGGAGCCGAAGTCAAGAAGCCCGGGTCGTC
AGTGAAAGTGTCCTGCAAGGCCTCCGGCGGAACCTTCAGCTCGTACTTCT
GGTCCTGGGTTAGGCAGGCCCCTGGACAAGGCCTCGAATGGATGGGTCGG
TTCTACTCCGGCGGTTCCGCCAACTACAACCCGTCACTGAAGGAGAGAGT
GACCATTACCGCGGATGAGTCGACTAGCACCGCCTACATGGAACTGTCCA
GCCTGCGGTCCGAGGACACTGCAGTCTACTATTGTGCTCGCGAGCGGCGC
GGGTACTCTGGCGGATTTGAAATCTGGGGACAGGGAACCCTGGTCACCGT
CTCCTCAGCCTCCACCAAGGGCCCATCGGTCTTCCCCCTGGCACCCTCCT
CCAAGAGCACCTCTGGGGGCACAGCGGCCCTGGGCTGCCTGGTCAAGGAC
TACTTCCCCGAACCGGTGACGGTGTCGTGGAACTCAGGCGCCCTGACCAG
CGGCGTGCACACCTTCCCGGCTGTCCTACAGTCCTCAGGACTCTACTCCC
TCAGCAGCGTGGTGACCGTGCCCTCCAGCAGOTTGGGCACCCAGACCTAC
ATCTGCAACGTGAATCACAAGCCCAGCAACACCAAGGTGGACAAGAAAGT
TGAGCCTAAGAGCTGCGGTGGCGGTGGATCACAAGTGCAGCTTGTGCAGA
GCGGAGCCGAAGTCAAGAAGCCCGGGTCGTCAGTGAAAGTGTCCTGCAAG
GCCTCCGGCGGAACCTTCAGCTCGTACTTCTGGTCCTGGGTTAGGCAGGC
CCCTGGACAAGGCCTCGAATGGATGGGTCGGTTCTACTCCGGCGGTTCCG
CCAACTACAACCCGTCACTGAAGGAGAGAGTGACCATTACCGCGGATGAG
TCGACTAGCACCGCCTACATGGAACTGTCCAGCCTGCGGTCCGAGGACAC
TGCAGTCTACTATTGTGCTCGCGAGCGGCGCGGGTACTCTGGCGGATTTG
AAATCTGGGGACAGGGAACCCTGGTCACCGTCTCCTCAGCCTCCACCAAG
GGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGT
GCCCTCCAGCAGCTTGGGCACCCAGACCTACATCTGCAACGTGAATCACA
AGCCCAGCAACACCAAGGTGGACAAGAAAGTTGAGCCCAAATCTTGTGAC
AAAACTCACACATGCCCACCGTGCCCAGCACCTGAAGCCGCTGGGGCACC
GTCAGTCTTCCTCTTCCCCCCAAAACCCAAGGACACCCTCATGATCTCCC
GGACCCCTGAGGTCACATGCGTGGTGGTGGACGTGAGCCACGAAGACCCT
GAGGTCAAGTTCAACTGGTACGTGGACGGCGTGGAGGTGCATAATGCCAA
GACAAAGCCGCGGGAGGAGCAGTACAACAGCACGTACCGTGTGGTCAGCG
TCCTCACCGTCCTGCACCAGGACTGGCTGAATGGCAAGGAGTACAAGTGC
AAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGA
GAACAACTACAAGACCACGCCTCCCGTGCTGGACTCCGACGGCTCCTTCT
TCCTCTATAGCAAGCTCACCGTGGACAAGAGCAGGTGGCAGCAGGGGAAC
GTCTTCTCATGCTCCGTGATGCATGAGGCTCTGCACAACCACTACACGCA
GAAGAGCCTCTCCCTGTCCCCCGGAAAA

In some embodiments, the disclosure provides polynucleotides encoding an anti-LTBR antibody light chain polypeptide comprising an amino acid sequence of SEQ ID NO: 13. In some embodiments, a polynucleotide comprising the nucleotide sequence of SEQ ID NO: 20 encodes a light chain polypeptide comprising the amino acid sequence of SEQ ID NO: 13.

### SEQ ID NO: 20 is:

(SEQ ID NO: 20)
GACATTGTGATGACTCAGTCGCCGTTGTCGCTGCCTGTGACTCCTGGAGA
GCCGGCCTCGATCTCCTGCCGGTCAAGCCAGTCCCTGCTGCACTCCAACT
CCTATAACTACCTGGATTGGTACCTCCAAAAGCCTGGGCAGAGCCCCCAG
CTCCTGATCTACCTTGGCTCTTACCGGGCCTCCGGAGTGCCGGACAGATT
CAGCGGTTCCGGATCAGGAACCGACTTTACCCTGAAAATCTCCCGCGTGG
AAGCGGAAGATGTCGGCGTGTACTACTGTATGCAGCCCCTGCAAACTCCA
TTCACCTTCGGGCCCGGCACCAAGGTCGACATCAAACGAACTGTGGCTGC
ACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAG
TGTCACAGAGCAGGACAGCAAGGACAGCACCTACAGCCTCAGCAGCACCC
TGACGCTGAGCAAAGCAGACTACGAGAAACACAAAGTCTACGCCTGCGAA
GTCACCCATCAGGGCCTGAGCTCGCCCGTCACAAAGAGCTTCAACAGGGG
AGAGTGT

In some embodiments, the disclosure provides polynucleotides encoding an anti-LTBR antibody VH polypeptide comprising an amino acid sequence of SEQ ID NO: 10. In some embodiments, a polynucleotide comprising the nucleotide sequence of SEQ ID NO: 21 encodes a VH polypeptide comprising the amino acid sequence of SEQ ID NO: 10.

### SEQ ID NO: 21 is:

(SEQ ID NO: 21)
CAAGTGCAGOTTGTGCAGAGCGGAGCCGAAGTCAAGAAGCCCGGGTCGTC
AGTGAAAGTGTCCTGCAAGGCCTCCGGCGGAACCTTCAGCTCGTACTTCT
GGTCCTGGGTTAGGCAGGCCCCTGGACAAGGCCTCGAATGGATGGGTCGG
TTCTACTCCGGCGGTTCCGCCAACTACAACCCGTCACTGAAGGAGAGAGT
GACCATTACCGCGGATGAGTCGACTAGCACCGCCTACATGGAACTGTCCA
GCCTGCGGTCCGAGGACACTGCAGTCTACTATTGTGCTCGCGAGCGGCGC
GGGTACTCTGGCGGATTTGAAATCTGGGGACAGGGAACCCTGGTCACCGT
CTCCTCA

In some embodiments, the disclosure provides polynucleotides encoding an anti-LTBR antibody VL polypeptide comprising an amino acid sequence of SEQ ID NO: 11. In some embodiments, a polynucleotide comprising the nucleotide sequence of SEQ ID NO: 22 encodes a VL polypeptide comprising the amino acid sequence of SEQ ID NO: 11.

### SEQ ID NO: 22 is:

(SEQ ID NO: 22)
GACATTGTGATGACTCAGTCGCCGTTGTCGCTGCCTGTGACTCCTGGAGA
GCCGGCCTCGATCTCCTGCCGGTCAAGCCAGTCCCTGCTGCACTCCAACT
CCTATAACTACCTGGATTGGTACCTCCAAAAGCCTGGGCAGAGCCCCCAG
CTCCTGATCTACCTTGGCTCTTACCGGGCCTCCGGAGTGCCGGACAGATT
CAGCGGTTCCGGATCAGGAACCGACTTTACCCTGAAAATCTCCCGCGTGG
AAGCGGAAGATGTCGGCGTGTACTACTGTATGCAGCCCCTGCAAACTCCA
TTCACCTTCGGGCCCGGCACCAAGGTCGACATCAAA

In some embodiments, the disclosure provides a polynucleotide or polynucleotides comprising one or more nucleotide sequences encoding any anti-LTBR antibody described herein, such as: i) an isolated antibody that binds to LTBR and comprises a VH and a VL wherein the VH comprises the amino acid sequence of SEQ ID NO: 10 and the VL comprises the amino acid sequence of SEQ ID NO: 11 ii) an isolated antibody comprising a light chain comprising the amino acid sequence of SEQ ID NO: 13 and a heavy chain comprising the amino acid sequence of SEQ ID NO: 12, wherein the C-terminal lysine of SEQ ID NO:12 is optional; and iii) an isolated antibody comprising a light chain comprising the amino acid sequence of SEQ ID NO: 13 and a heavy chain comprising the amino acid sequence of SEQ ID NO: 14, wherein the C-terminal lysine of SEQ ID NO:14 is optional.

In some embodiments, provided herein is a polynucleotide comprising the nucleic acid sequence of the insert of the plasmid deposited with the ATCC and having Accession No. PTA-127515 encoding the VH of antibody 1E01. In some embodiments, provided herein is a polynucleotide comprising the nucleic acid sequence of the insert of the plasmid deposited with the ATCC and having Accession No. PTA-127516 encoding the VL of antibody 1E01.

In addition, also provided herein is a polypeptide comprising the amino acid sequence encoded by the DNA insert of the plasmid deposited with the ATCC and having Accession No. PTA-127515, encoding the VH domain of antibody 1E01. Also provided herein is a polypeptide comprising the amino acid sequence encoded by the insert of the plasmid deposited with the ATCC and having Accession No. PTA-127516 encoding the VL domain of antibody 1E01.

In some embodiments, provided herein is an antibody comprising a VH encoded by the DNA insert of the plasmid deposited with the ATCC and having Accession No. PTA-127515 and a VL encoded by the DNA insert of the plasmid deposited with the ATCC and having Accession No. PTA-127516. In some embodiments, provided herein is an antibody comprising a VH encoded by the DNA insert of the plasmid deposited with the ATCC and having Accession No. PTA-127515 and a VL encoded by the DNA insert of the plasmid deposited with the ATCC and having Accession No. PTA-127516, wherein the antibody is in bivalent or tetravalent format.

It will be appreciated by those of ordinary skill in the art that, as a result of the degeneracy of the genetic code, there are many nucleotide sequences that encode a polypeptide as described herein. Some of these polynucleotides bear minimal homology to a nucleotide sequence provided herein. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection. F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

### Pharmaceutical Compositions

In another embodiment, the invention comprises pharmaceutical compositions.

A “pharmaceutical composition” refers to a mixture of an antibody the invention and one or excipient.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences. Mack Publishing Co., Easton, Pennsylvania, 1975: Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

### Therapeutic, Diagnostic, and Other Methods

Antibodies of the present invention are useful in various applications including but not limited to, as a medicament, for therapeutic treatment methods and for diagnostic treatment methods.

In some embodiments, antibodies of the invention may agonize the activity of LTBR and may be useful in the treatment of diseases that can be treated by increased LTBR activity (e.g. cancer).

In one aspect, the invention provides a method for treating cancer. In some embodiments, the method of treating cancer in a subject comprises administering to the subject in need thereof an effective amount of a pharmaceutical composition comprising any of the anti-LTBR antibodies as described herein. In some embodiments, provided herein is a method of treating cancer in a subject, comprising administering to the subject in need thereof an effective amount of a composition comprising an antibody provided herein.

In some embodiments, provided herein is a method of inducing the formation or maturation of tertiary lymphoid structures (TLS) in a subject, comprising administering to the subject in need thereof an effective amount of a composition comprising an anti-LTBR antibody provided herein.

In some embodiments, provided herein is a method of inhibiting the growth of PD(L)1 inhibitor treatment resistant cancer cells in a subject, comprising administering to the subject in need thereof an effective amount of a composition comprising an anti-LTBR antibody provided herein.

In some embodiments, provided herein is a method of inhibiting the growth of cancer cells in a subject wherein the cancer or subject was previously treated with a PD(L)1 inhibitor, comprising administering to the subject in need thereof an effective amount of a composition comprising an anti-LTBR antibody provided herein.

In some embodiments, provided herein is a method of promoting the infiltration of CD8 positive (CD8+) T cells in the tumor microenvironment in a subject, comprising administering to the subject in need thereof an effective amount of a composition comprising an anti-LTBR antibody provided herein.

In some embodiments, provided herein is a method of promoting the production of CXCL13 in a subject, comprising administering to the subject in need thereof an effective amount of a composition comprising an anti-LTBR antibody provided herein.

In another aspect, the invention further provides an anti-LTBR antibody or pharmaceutical composition as described herein for use in the described method of treating cancer. The invention also provides the use of an antibody as described herein in the manufacture of a medicament for treating cancer.

In some embodiments, cancers that can be treated with anti-LTBR antibodies provided herein include, for example, bladder cancer, breast cancer, clear cell kidney cancer, head/neck squamous cell carcinoma (HNSCC) [squamous cell carcinoma of the head and neck (SCCHN)], lung squamous cell carcinoma, lung adenocarcinoma, malignant melanoma, non-small-cell lung cancer (NSCLC), ovarian cancer, pancreatic cancer, prostate cancer, renal cell carcinoma (RCC), small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), endometrial cancer, B-cell acute lymphoblastic leukemia, colorectal cancer (CRC), glioblastoma, uterine cancer, cervical cancer, penile cancer, gastric cancer (GC), and non-melanoma skin cancer, including such cancers that are PD(L)1 inhibitor resistant or refractory. In some embodiments, the cancer treated with anti-LTBR antibodies provided herein is melanoma, HNSCC, urothelial cancer, lung cancer (e.g. NSCLC), RCC, gastric cancer, CRC, cervical cancer, ovarian cancer, or breast cancer, including such cancers that are PD(L)1 inhibitor resistant or refractory (R/R). In some embodiments, the cancer treated with anti-LTBR antibodies provided herein is anti-PD(L)1 naïve (i.e. previously untreated) microsatellite stable (MSS) colorectal cancer, NSCLC, or urothelial cancer. In some embodiments, the cancer treated with anti-LTBR antibodies provided herein is PD(L)1 inhibitor R/R NSCLC, urothelial cancer, or melanoma.

In another aspect, provided is a method of one or more of detecting, diagnosing, or monitoring LTBR protein in a subject or a sample. For example, the anti-LTBR antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent.

With respect to all methods described herein, reference to anti-LTBR antibodies also includes pharmaceutical compositions comprising the anti-LTBR antibodies and one or more additional agents.

### Administration and Dosing

Typically, an antibody of the invention is administered in an amount effective to treat a condition as described herein. The antibodies the invention can be administered as an antibody per se, or alternatively, as a pharmaceutical composition containing the antibody.

The antibodies of the invention are administered by any suitable route in the form of a pharmaceutical composition adapted to such a route, and in a dose effective for the treatment intended.

In some embodiments, the antibodies may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques. In some embodiments, an anti-LTBR antibody provided herein is administered subcutaneously (SC). In some embodiments, an anti-LTBR antibody provided herein is administered intravenously (IV).

The dosage regimen for the antibodies of the invention or compositions containing said antibodies is based on a variety of factors, including the type, age, weight, sex and medical condition of the subject; the severity of the condition; the route of administration; and the activity of the particular antibody employed. Thus, the dosage regimen may vary widely. In one embodiment, the total daily dose of an antibody of the invention is typically from about 0.01 to about 100 mg/kg (i.e., mg antibody of the invention per kg body weight) for the treatment of the indicated conditions discussed herein. In another embodiment, total daily dose of the antibody of the invention is from about 0.1 to about 50 mg/kg, and in another embodiment, from about 0.5 to about 30 mg/kg.

In some embodiments, an anti-LTBR antibody provided herein is administered once a week (Q1W), once every two weeks (Q2W), once every three weeks (Q3W), once every four weeks (Q4W), once every five weeks (Q5W), once every six weeks (Q6W), once a month (Q1M), once every two months (Q2M), or once every three months (Q3M).

In some embodiments, an anti-LTBR antibody provided at a dose of about 1 mg/kg, 2 mg/kg, 3 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg.

In some embodiments, an anti-LTBR antibody provided IV Q1W at a dose of about 5 mg/kg, 10 mg/kg, 15 mg/kg, 20 mg/kg or 25 mg/kg. In some embodiments, an anti-LTBR antibody provided IV Q2W at a dose of about 15 mg/kg, 20 mg/kg, 25 mg/kg, 30 mg/kg, or 35 mg/kg.

In some embodiments, an anti-LTBR antibody provided SC 01W at a dose of about 15 mg/kg, 20 mg/kg, 25 mg/kg, 30 mg/kg, or 35 mg/kg. In some embodiments, an anti-LTBR antibody provided SC 02W at a dose of about 40 mg/kg, 45 mg/kg, 50 mg/kg, 55 mg/kg, or 60 mg/kg.

### Co-Administration

The antibodies of the invention can be used alone, or in combination with one or more other therapeutic agents. The invention provides any of the uses, methods or compositions as defined herein wherein an antibody of the invention is used in combination with one or more other therapeutic agent discussed herein.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

In some embodiments, an anti-LTBR antibody provided herein may be administered in combination with the administration of one or more additional therapeutic agents. Optionally, the additional therapeutic agent may include an additional anti-cancer agent. These include, but are not limited to, the administration of a biotherapeutic agent and/or a chemotherapeutic agent, such as but not limited to, a vaccine, a CAR-T cell-based therapy, radiotherapy, a cytokine therapy, a CD3 bispecific antibody, an inhibitor of other immunosuppressive pathways, an inhibitor of angiogenesis, a T cell activator, an inhibitor of a metabolic pathway, an mTOR inhibitor, an inhibitor of an adenosine pathway, a tyrosine kinase inhibitor including but not limited to Inlyta, ALK inhibitors and sunitinib, a BRAF inhibitor, an epigenetic modifier, an IDO1 inhibitor, a JAK inhibitor, a STAT inhibitor, a cyclin-dependent kinase inhibitor, a biotherapeutic agent (including but not limited to antibodies to VEGF, VEGFR, EGFR, Her2/neu, other growth factor receptors, CD40, CD-40L, CTLA-4, OX-40, 4-1BB, TIGIT, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2. GM-CSF), and cells transfected with genes encoding immune stimulating cytokines such as but not limited to GM-CSF).

Examples of biotherapeutic agents include therapeutic antibodies, immune modulating agents, and therapeutic immune cells.

Therapeutic antibodies may have specificity against a variety of different of antigens. For example, therapeutic antibodies may be directed to a tumor associated-antigen, such that binding of the antibody to the antigen promotes death of the cell expressing the antigen. In other example, therapeutic antibodies may be directed to an antigen (e.g. PD-1) on an immune cell, such that binding of the antibody prevents downregulation of the activity of the cell expressing the antigen (and thereby promotes activity of the cell expressing the antigen). In some situations, a therapeutic antibody may function through multiple different mechanisms (for example, it may both i) promote death of the cell expressing the antigen, and ii) prevent the antigen from causing down-regulation of the activity of immune cells in contact with the cell expressing the antigen).

Therapeutic antibodies may be directed to, for example, the antigens listed as follows. For some antigens, exemplary antibodies directed to the antigen are also included below (in brackets/parenthesis after the antigen). The antigens as follow may also be referred to as “target antigens” or the like herein. Target antigens for therapeutic antibodies herein include, for example: 4-1BB (e.g. utomilumab); 5T4; A33; alpha-folate receptor 1 (e.g. mirvetuximab soravtansine); Alk-1; BCMA [e.g. PF-06863135 (see U.S. Pat. No. 9,969,809)]; BTN1A1 (e.g. see WO2018222689); CA-125 (e.g. abagovomab); Carboanhydrase IX; CCR2; CCR4 (e.g. mogamulizumab); CCR5 (e.g. leronlimab); CCR8: CD3 [e.g. blinatumomab (CD3/CD19 bispecific), PF-06671008 (CD3/P-cadherin bispecific), PF-06863135 (CD3/BCMA bispecific), CD19 (e.g. blinatumomab, MOR208); CD20 (e.g. ibritumomab tiuxetan, obinutuzumab, ofatumumab, rituximab, ublituximab); CD22 (inotuzumab ozogamicin, moxetumomab pasudotox); CD25; CD28; CD30 (e.g. brentuximab vedotin); CD33 (e.g. gemtuzumab ozogamicin); CD38 (e.g. daratumumab, isatuximab), CD40; CD-40L; CD44v6; CD47; CD52 (e.g. alemtuzumab); CD63; CD79 (e.g. polatuzumab vedotin); CD80; CD123; CD276/B7-H3 (e.g. omburtamab); CDH17; CEA; CIhCG; CTLA-4 (e.g. ipilimumab, tremelimumab), CXCR4; desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvIII; Endosialin: EpCAM (e.g. oportuzumab monatox): FAP: Fetal Acetylcholine Receptor; FLT3 (e.g. see WO2018/220584); GD2 (e.g. dinutuximab, 3F8); GD3; GITR; GloboH; GM1; GM2; GUCY2C (e.g. PF-07062119); HER2/neu [e.g. margetuximab, pertuzumab, trastuzumab; ado-trastuzumab emtansine, trastuzumab duocarmazine, PF-06804103 (see U.S. Pat. No. 8,828,401)]; HER3; HER4; ICOS; IL-10; ITG-AvB6; LAG-3 (e.g. relatlimab); Lewis-Y: LG; Ly-6; M-CSF [e.g. PD-0360324 (see U.S. Pat. No. 7,326,414)]; MCSP: mesothelin; MUC1; MUC2; MUC3; MUC4; MUCSAC; MUC5B; MUC7; MUC16; Notch1; Notch3; Nectin-4 (e.g. enfortumab vedotin); OX40 [e.g. PF-04518600 (see U.S. Pat. No. 7,960,515)]; P-Cadherin [e.g. PF-06671008 (see WO2016/001810)]; PCDHB2; PD-1 [e.g. BCD-100, camrelizumab, cemiplimab, genolimzumab (CBT-501), MED10680, nivolumab, pembrolizumab, RN888 (see WO2016/092419), sintilimab, spartalizumab, STI-A1110, tislelizumab. TSR-042]; PD-L1 (e.g. atezolizumab, durvalumab, BMS-936559 (MDX-1105), or LY3300054); PDGFRA (e.g. olaratumab); Plasma Cell Antigen; PolySA; PSCA; PSMA; PTK7 [e.g. PF-06647020 (see U.S. Pat. No. 9,409,995)]; Ror1; SAS; SCRx6; SLAMF7 (e.g. elotuzumab); SHH; SIRPa (e.g. ED9, Effi-DEM); STEAP; TGF-beta; TIGIT; TIM-3; TMPRSS3; TNF-alpha precursor; TROP-2 (e.g sacituzumab govitecan); TSPAN8; VEGF (e.g. bevacizumab, brolucizumab); VEGFR1 (e.g. ranibizumab); VEGFR2 (e.g. ramucirumab, ranibizumab); Wue-1.

Therapeutic antibodies administered in combination with the antibodies provided herein may have any suitable format. For example, therapeutic antibodies may have any format as described elsewhere herein. In some embodiments, a therapeutic antibody may be a naked antibody. In some embodiments, a therapeutic antibody may be linked to a drug or other agent (also known as an “antibody-drug conjugate” (ADC)). In some embodiments, a therapeutic antibody against a particular antigen may incorporated into a multi-specific antibody (e.g. a bispecific antibody).

In some embodiments, an anti-LTBR antibody provided herein may be administered in combination with a pattern recognition receptor (PRR) agonists, immunostimulatory cytokines, and cancer vaccines. There are multiple classes of PRR molecules, including toll-like receptors (TLRs). RIG-1-like receptors (RLRs), nucleotide-binding oligomerization domain (NOD)-like receptors (NLRs), C-type lectin receptors (CLRs), and Stimulator of Interferon Genes (STING) protein. Other PRRs include, for example, DNA-dependent Activator of IFN-regulatory factors (DAI) and Absent in Melanoma 2 (AIM2). In some embodiments, an anti-LTBR antibody provided herein may be administered in combination with a TLR agonist (e.g. TLR2, TLR3. TLR4, TLR5, TLR6, TLR7, TLR8, or TLR9 agonist).

Examples of immunostimulatory cytokines that are useful in the treatment methods, medicaments, and uses of the present invention include GM-CSF, G-CSF, IFN-alpha, IFN-gamma; IL-2 (e.g. denileukin difitox), IL-6, IL-7, IL-11, IL-12, IL-15, IL-18, IL-21, and TNF-alpha.

Examples of cancer vaccines that are useful in the treatment methods, medicaments, and uses of the present invention include, for example, sipuleucel-T and talimogene laherparepvec (T-VEC).

Examples of immune cell therapies that are useful in the treatment methods, medicaments, and uses of the present invention include, for example, tumor-infiltrating lymphocytes (TILs) and chimeric antigen receptor T cells (CAR-T cells).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g., calicheamicin, especially calicheamicin gamma1l and calicheamicin phi1l, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-nordeucine, doxorubicin (including morpholino-doxorubicin, cyanomorpholino-doxorubicin, 2-pyrrolino-doxorubicin, and deoxydoxorubicin), pegylated liposomal doxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, rordin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil: gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; KRAS inhibitors: MCT4 inhibitors; MAT2a inhibitors; tyrosine kinase inhibitors such as sunitinib, axitinib; alk/c-Met/ROS inhibitors such as crizotinib, lorlatinib; mTOR inhibitors such as temsirolimus, gedatolisib; src/abl inhibitors such as bosutinib; cyclin-dependent kinase (CDK) inhibitors such as palbociclib, PF-06873600; erb inhibitors such as dacomitinib; PARP inhibitors such as talazoparib; SMO inhibitors such as glasdegib, PF-5274857; EGFR T790M inhibitors such as PF-06747775; EZH2 inhibitors such as PF-06821497; PRMT5 inhibitors such as PF-06939999; TGFRPr1 inhibitors such as PF-06952229; and pharmaceutically acceptable salts, acids or derivatives of any of the above. In specific embodiments, such additional therapeutic agent is bevacizumab, cetuximab, sirolimus, panitumumab, 5-fluorouracil (5-FU), capecitabine, tivozanib, irinotecan, oxaliplatin, cisplatin, trifluidine, tipiracil, leucovorin, gemcitabine, regorafinib or erlotinib hydrochloride.

In some embodiments, an anti-LTBR antibody provided herein is administered in combination with a PD1 or PDL1 inhibitor. PD1 and PDL1 inhibitors are collectively referred to herein as “PD(L)1” inhibitors. In some embodiments, an anti-LTBR antibody provided herein administered in combination with an VEGF or VEGF receptor (VEGFR) inhibitor. VEGF and VEGFR inhibitors are collectively referred to herein as “VEGF(R)” inhibitors. VEGF(R) inhibitors include agents that bind to any VEGF subtype (e.g. VEGF-A, VEGF-C, and VEGF-D) and VEGFR subtype (e.g. VEGFR1, VEGFR2, and VEGFR3). In some embodiments, an anti-LTBR antibody provided herein is administered in combination with both a PD(L)1 inhibitor and a VEGF(R) inhibitor. In some embodiments, the PD(L)1 inhibitor is sasanlimab. In some embodiments, the VEGF(R) inhibitor is axitinib or bevacizumab.

In some embodiments, PD(L)1 inhibitors are anti-PD1 or anti-PD-L1 antibodies. These bind to PD1 or PDL1, and block the interaction between PD1 and PDL1. Examples of PD(L)1 inhibitors useful in methods, medicaments and uses of the present invention include, for example: sasanlimab (aka RN888, an anti-PD1 IgG4 monoclonal antibody) pembrolizumab (aka MK-3475, an anti-PD-1 IgG4 monoclonal antibody) nivolumab (aka BMS-936558 or MDX1106, an anti-PD-1 IgG4 monoclonal antibody), cemiplimab (aka REGN-2810, an anti-PD-1 antibody), atezolizumab (aka MPDL3280A an IgG1-engineered, anti-PD-L1 antibody), BMS-936559 (a fully human, anti-PD-L1, IgG4 monoclonal antibody), MED14736 (aka durvalumab, an engineered IgG1 kappa anti-PD-L1 monoclonal antibody with triple mutations in the Fc domain to remove antibody-dependent, cell-mediated cytotoxic activity). Additional exemplary PD(L)1 inhibitors useful in the methods, medicaments and uses of the present invention include SHR1210 (anti-PD-1 antibody), KN035 (anti-PD-L1 antibody), IBI308 (anti-PD-1 antibody), PDR001 (anti-PD-1 antibody), BGB-A317 (anti-PD-1 antibody), BCD-100 (anti-PD-1 antibody), JS001 (anti-PD-1 antibody). In some embodiments, a PD(L)1 inhibitor is a small molecule PD-1 or PD-L1 antagonist (e.g. CA-170), as described in Yang et al Med. Res. Rev. (2019), 39, pp 265-301.

In some embodiments, VEGF(R) inhibitors are anti-VEGF or anti-VEGFR antibodies. These bind to VEGF or VEGFR, and block the interaction between VEGF and VEGFR and/or inhibit the activity of VEGFR. Anti-VEGF(R) antibodies include, for example, bevacizumab, ramucirumab, and ranibizumab. In some embodiments, VEGF(R) inhibitors are small molecule agents that bind to VEGF or VEGFR and inhibit the activity of VEGFR. Small molecule VEGF(R) inhibitors include, for example, apatinib, axitinib, cabozantinib, lapatinib, lenvatinib, nintedanib, pazopanib, ponatinib, regorafenib, sorafenib, sunitinib, and vandetanib.

In some embodiments, an anti-LTBR antibody provided herein is administered in combination with a PD(L)1 inhibitor to treat urothelial cancer, NSCLC, or melanoma. In some embodiments, an anti-LTBR antibody provided herein is administered in combination with a VEGF axis inhibitor to treat NSCLC or colorectal cancer (for example, microsatellite stable (MSS) colorectal cancer).

In some embodiments, an anti-LTBR antibody therapy may be co-administered with, or be sequentially administered before or after the other agent treatment by intervals ranging from minutes to weeks. In embodiments where the other agents and/or a proteins or polynucleotides are administered separately, one would generally ensure that a significant period of time did not expire between each delivery, such that the agent and the composition of the present invention would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

In some embodiments, an anti-LTBR antibody therapy composition is combined with a treatment regimen further comprising a traditional therapy selected from the group consisting of: surgery, radiation therapy, chemotherapy, targeted therapy, immunotherapy, hormonal therapy, angiogenesis inhibition and palliative care.

### Kits

Another aspect of the invention provides kits comprising an anti-LTBR antibody of the invention or pharmaceutical compositions comprising the antibody. A kit may include, in addition to an antibody of the invention or pharmaceutical composition thereof, diagnostic or therapeutic agents. A kit may also include instructions for use in a diagnostic or therapeutic method. In some embodiments, the kit includes an antibody or a pharmaceutical composition thereof and a diagnostic agent. In other embodiments, the kit includes an antibody or a pharmaceutical composition thereof and one or more therapeutic agents, such as a PD(L)1 inhibitor or VEGF(R) inhibitor.

In yet another embodiment, the invention comprises kits that are suitable for use in performing the methods of treatment described herein. In one embodiment, the kit contains a first dosage form comprising one or more of the antibodies of the invention in quantities sufficient to carry out the methods of the invention. In another embodiment, the kit comprises one or more antibodies of the invention in quantities sufficient to carry out the methods of the invention and at least a first container for a first dosage and a second container for a second dosage.

### Biological Deposits

Representative materials of the present invention were deposited in the American Type Culture Collection (ATCC), 10801 University Boulevard, Manassas, VA 20110-2209, USA, on Feb. 7, 2023. Vector “1E01_VH” having ATCC Accession No. PTA-127515 comprises a DNA insert encoding the anti-LTBR VH designated “1E01” and vector “1E01_VL” having ATCC Accession No. PTA-127516 comprises a DNA insert encoding the anti-LTBR VL designated “1E01”.

| Antibody | Description | ATCC Accession No. |
|---|---|---|
| 1E01 | 1E01_VH (VH of antibody 1E01) | PTA-127515 |
| 1E01 | 1E01_VL (VL of antibody 1E01) | PTA-127516 |

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions; the materials will be promptly replaced on notification with another of the same. Availability of the deposited material is not to be construed as a license to practice the invention in contravention of the rights granted under the authority of any government in accordance with its patent laws

Incorporated by reference herein for all purposes is the content of U.S. Provisional Patent Application No. 63/340,753, filed May 11, 2022.

The following examples of specific aspects for carrying out the present invention are offered for illustrative purposes only, and are not intended to limit the scope of the present invention in any way.

The foregoing description and following Examples detail certain specific embodiments of the disclosure and describes the best mode contemplated by the inventors.

It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### EXAMPLES

In order that this invention may be better understood, the following examples are set forth. These examples are for purposes of illustration only and are not to be construed as limiting the scope of the invention in any manner.

### Example 1: Exemplary Anti-LTBR Antibodies

A multi-step screening process was used to generate and identify antibodies with high affinity for human LTBR. Starting from around 40 million fused lymphocytes, a small number (˜10) lead candidate anti-LTBR clones were identified. These lead candidates were further subjected to i) a series of functional assays including for NFkappaBeta (NFkB) activation and induction of Cxcl10, ICAM-1, and IL-8, and ii) computational analysis of predicted immunogenicity.

Based on these assays, the Fab clone “1E01” was identified as having the best combination of i) LTBR binding and agonist activity, and ii) low immunogenicity in humans. Table 4 provides sequence information for the 1E01 clone. Complementarity-determining regions (CDRs) are in bold using Chothia numbering and underlined using Kabat numbering.

TABLE 4

| ID | Sequence |
|---|---|
| 1E01 | QVQLVQSGAEVKKPGSSVKVSCKASGGTFSSYFWSWVRQAPGQG |
| VH | LEWMGRFYSGGSANYNPSLKERVTITADESTSTAYMELSSLRSE |
| | DTAVYYCARERRGYSGGFEIWGQGTLVTVSS (SEQ ID NO: |
| | 10) |
| 1E01 | DIVMTQSPLSLPVTPGEPASISCRSSQSLLHSNSYNYLDWYLQK |
| VL | PGQSPQLLIYLGSYRASGVPDRFSGSGSGTDFTLKISRVEAEDV |
| | GVYYCMQPLQTPFTFGPGTKVDIK (SEQ ID NO: 11) |

Anti-LTBR clone 1E01 has a similar binding affinity (K_{D}) to human LTBR and cynomolgus monkey LTBR, having a K_{D} of 115+/−26 nM to human LTBR, and a K_{D} of 125+/−35 nM to cynomolgus monkey LTBR. No binding to mouse LTBR was detected.

In binning assays, 1E01 was found to bind to an epitope on the LTBR cysteine rich domain one (CRD1) (amino acids 62-102).

1E01 does not block ligand (e.g. LIGHT or LTa1b2) binding to LTBR.

### LTBR Epitope Analysis

To identify the epitope on LTBR to which the 1E01 antibody binds, it was initially attempted to crystalize the 1E01 antibody in complex with human LTBR protein. However, these efforts did not yield suitable crystals for structural analysis.

Next, a “Fab sandwich” strategy was used for crystallization in which the 1E01 antibody and another anti-LTBR clone (2C09) were used together with human LTBR protein. The 2C09 and 1E01 anti-LTBR antibodies have non-overlapping epitopes as determined by binning data. Crystallization of the LTBR-1E01-2C09 Fab sandwich complex was successful, with the entire LTBR extracellular domain ordered in the crystal.

The structure indicated that the 1E01 and 2C09 antibodies bind on opposite faces of the LTBR CRD1 and CRD2 domains. In addition, in the crystal structure the following human LTBR residues are within 3.8 angstroms of the 1E01 Fab: P63, P64, G65, T66, Y67, S69, A70, R76, T78, V79, C80, T82, C83, A84, E85, W92, K119, thus indicating that they are amino acids of the epitope on LTBR bound by 1E01. These amino acid residues are underlined and bold in the human LTBR amino acid sequence (SEQ ID NO: 15) as shown below:

(SEQ ID NO: 15)
MLLPWATSAPGLAWGPLVLGLFGLLAASQPQAVPPYASENQTCRDQEKEY
YEPQHRICCSRCPPGTYVSAKCSRIRDTVCATCAENSYNEHWNYLTICQL
CRPCDPVMGLEEIAPCTSKRKTQCRCQPGMFCAAWALECTHCELLSDCPP
GTEAELKDEVGKGNNHCVPCKAGHFQNTSSPSARCQPHTRCENQGLVEAA
PGTAQSDTTCKNPLEPLPPEMSGTMLMLAVLLPLAFFLLLATVFSCIWKS
HPSLCRKLGSLLKRRPQGEGPNPVAGSWEPPKAHPYFPDLVQPLLPISGD
VSPVSTGLPAAPVLEAGVPQQQSPLDLTREPQLEPGEQSQVAHGTNGIHV
TGGSMTITGNIYIYNGPVLGGPPGPGDLPATPEPPYPIPEEGDPGPPGLS
TPHQEDGKAWHLAETEHCGATPSNRGPRNQFITHD.

The residues P63, P64, G65, T66, Y67, S69, A70, R76, T78, V79, C80, T82, C83, A84, E85, W92 are in LTBR CRD1; the residue K119 is in LTBR CRD2.

### Example 2: Comparison of Antibody Formats

In this example, activity of the 1E01 antibody in bivalent and tetravalent formats was examined.

For the bivalent antibody, the 1E01 VH and VL were introduced into a standard human IgG1 format. A schematic of this antibody is shown in FIG. 1A. The bivalent antibody has two heavy chains and two light chains. Each heavy chain has, in N-terminus to C-terminus order, a VH, CH1, CH2, and CH3 domain, and the CH1 and CH2 domains are separated by a hinge region. There are two antigen binding sites in this molecule (the two VHNL pairs). The heavy chain of the antibody comprises the amino acid sequence of SEQ ID NO: 14 and the light chain comprises the amino sequence of SEQ ID NO: 13.

| ID | Sequence |
|---|---|
| 1E01 | QVQLVQSGAEVKKPGSSVKVSCKASGGTFSSYFWSWVRQAPGQGLEWMGRFYS |
| Bivalent | GGSANYNPSLKERVTITADESTSTAYMELSSLRSEDTAVYYCARERRGYSGGF |
| Heavy | EIWGQGTLVTVSSASTKGPSVFPLAPSSKSTSGGTAALGCLVKDYFPEPVTVS |
| chain | WNSGALTSGVHTFPAVLQSSGLYSLSSWVTVPSSSLGTQTYICNVNHKPSNTK |
| | VDKKVEPKSCDKTHTCPPCPAPELLGGPSVFLFPPKPKDTLMISRTPEVTCVW |
| | DVSHEDPEVKFNWYVDGVEVHNAKTKPREEQYNSTYRVVSVLTVLHQDWLNGK |
| | EYKCKVSNKALPAPIEKTISKAKGQPREPQVYTLPPSREEMTKNQVSLTCLVK |
| | GFYPSDIAVEWESNGQPENNYKTTPPVLDSDGSFFLYSKLTVDKSRWQQGNVF |
| | SCSVMHEALHNHYTQKSLSLSPGK (SEQ ID NO: 14) |
| 1E01 | DIVMTQSPLSLPVTPGEPASISCRSSQSLLHSNSYNYLDWYLQKPGQSPQLLI |
| Bivalent | YLGSYRASGVPDRFSGSGSGTDFTLKISRVEAEDVGVYYCMQPLQTPFTFGPG |
| Light | TKVDIKRTVAAPSVFIFPPSDEQLKSGTASVVCLLNNFYPREAKVQWKVDNAL |
| chain | QSGNSQESVTEQDSKDSTYSLSSTLTLSKADYEKHKVYACEVTHQGLSSPVTK |
| | SFNRGEC (SEQ ID NO: 13) |

For the tetravalent antibody, the 1E01 VH and VL were introduced into an extended length heavy chain having double the amount of VH and CHT domains as an in a standard human IgG G heavy chain. A schematic of this antibody is shown in FIG. VB. The tetravalent antibody has two extended heavy chains, and four light chains. Each extended heavy chain has, in N-terminus to C-terminus order, a VH, CH1, VH. CH1, CH2, CH3 domain, and the CH1 and CH2 domains are separated by a hinge region. The first CH1 is separated from the second VH by a short linker polypeptide, having the amino acid sequence EPKSCGGGGS (SEQ ID NO: 18). There are four antigen binding sites in this molecule (the four VH/VL pairs). Each heavy chain of the antibody comprises the amino acid sequence of SEQ ID NO: 12 and each light chain comprises the amino sequence of SEQ ID NO: 13. The heavy chain of SEQ ID NO: 12 also contains mutations in the IgG1 CH2 domain (1L234A, L235A, and G237A; EU numbering) to reduce IgG1-Fc domain effector function.

| ID | Sequence |
|---|---|
| 1E01 | QVQLVQSGAEVKKPGSSVKVSCKASGGTFSSYFWSWVRQAPGQGLEWMGRFYS |
| Tetra- | GGSANYNPSLKERVTITADESTSTAYMELSSLRSEDTAVYYCARERRGYSGGF |
| valent | EIWGQGTLVTVSSASTKGPSVFPLAPSSKSTSGGTAALGCLVKDYFPEPVTVS |
| Heavy | WNSGALTSGVHTFPAVLQSSGLYSLSSVVTVPSSSLGTQTYICNVNHKPSNTK |
| chain | VDKKVEPKSCGGGGSQVQLVQSGAEVKKPGSSVKVSCKASGGTFSSYFWSWVR |
| | QAPGQGLEWMGRFYSGGSANYNPSLKERVTITADESTSTAYMELSSLRSEDTA |
| | VYYCARERRGYSGGFEIWGQGTLVTVSSASTKGPSVFPLAPSSKSTSGGTAAL |
| | GCLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPEAAGAPSVFLFPPKPK |
| | DTLMISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLPPS |
| | REEMTKNQVSLTOLVKGFYPSDIAVEWESNGQPENNYKTTPPVLDSDGSFFLY |
| | SKLTVDKSRWQQGNVFSCSVMHEALHNHYTQKSLSLSPGK (SEQ ID NO: |
| | 12) |
| 1E01 | DIVMTQSPLSLPVTPGEPASISCRSSQSLLHSNSYNYLDWYLQKPGQSPQLLI |
| Tetra- | YLGSYRASGVPDRFSGSGSGTDFTLKISRVEAEDVGVYYCMQPLQTPFTFGPG |
| valent | TKVDIKRTVAAPSVFIFPPSDEQLKSGTASVVCLLNNFYPREAKVQWKVDNAL |
| Light | QSGNSQESVTEQDSKDSTYSLSSTLTLSKADYEKHKVYACEVTHQGLSSPVTK |
| chain | SFNRGEC (SEQ ID NO: 13) |

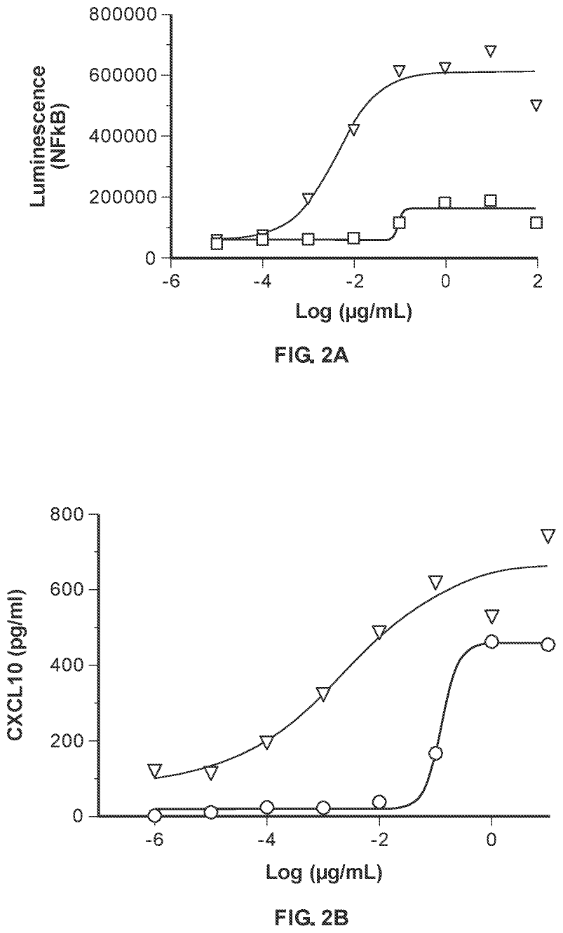

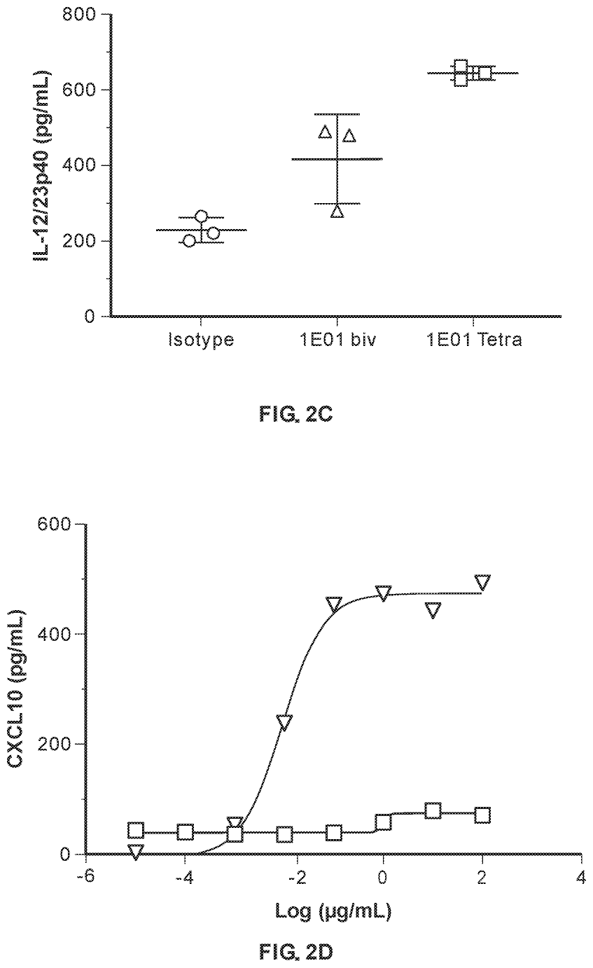

The bivalent and tetravalent 1E01 antibodies were compared in a panel of assays to assess LTBR-mediated downstream signaling. The assays were for: A) NFkappaBeta (NFkB) activation (using a luciferase reporter assay in a LTBR-expressing HEK293 cell) (FIG. 2A); B) CXCL10 induction in primary human fibroblasts (FIG. 2B); C) IL-12 induction in primary human monocyte-derived dendritic cells (moDC)(FIG. 2C); and D) CXCL10 induction in primary cynomolgus monkey fibroblasts (FIG. 2D). The assays for FIGS. 2A, 2B, and 2D were performed with a range of concentrations of each antibody as shown on each X axis (the assay of FIG. 2C used a single concentration for each antibody). For the assays, the cells were incubated with the antibodies for 2 days. Cytokines were read out via ELISA.

In each of FIGS. 2A and 2D, the data points for the tetravalent antibody are depicted with an inverted triangle and for the bivalent antibody are depicted with a square. In FIG. 2B, the data points for the tetravalent antibody are depicted with an inverted triangle and for the bivalent antibody are depicted with a circle. In FIG. 2C, the X-axis is labeled with the different antibodies (from left to right: isotype IgG1 (control); 1E01 bivalent; 1E01 tetravalent); the Y-axis depicts picograms/mL of the IL-12 (and IL-23) p40 subunit (p40 is a common subunit of IL-12 and IL-23). In FIGS. 2A, 2B, and 2D, a range of concentrations of each antibody was used as shown on the X-axis.

As shown in each of FIGS. 2A-2D, the tetravalent 1E01 antibody had a stronger LTBR activation activity than the bivalent antibody for each of the tested assays, as shown by greater NFkB activation (FIG. 2A), greater CXCL10 induction in primary human fibroblasts (FIG. 2B), greater IL-12 induction in primary human monocyte-derived dendritic cells (FIG. 2C), and greater CXCL10 induction in primary cynomolgus monkey fibroblasts (FIG. 2D).

### Example 3: Selection and Assessment of a Mouse Surrogate Anti-LTBR Antibody

As noted in Example 1, the 1E01 antibody has no detectable binding to mouse LTBR. Thus, to perform experiments in mouse models, a mouse surrogate for the 1E01 antibody was developed.

Antibodies were raised against mouse LTBR, and after a lengthy screening process the anti-mouse LTBR clone 5A7-N103A was selected as a mouse surrogate for the tetravalent 1E01 antibody. 5A7-N103A is a tetravalent antibody; the parent Fab is the “5A7” clone. 5A7-N103A was identified as the best representative mouse surrogate antibody for 1E01 based on: 1) partially overlapping epitope binding site on LTBR: 2) structural modeling indicative of similar binding geometry and LTBR clustering distance mediated by the antibody; 3) similar affinity of 5A7 for mouse LTBR as compared to 1E01 for human LTBR; and 4) biological functional assays indicative of similar in vitro activity. Like the tetravalent 1E01 antibody, the mouse 5A7-N103A also has mutations to reduce Fc domain effector function.

Additional details about these and other features of the 5A7-N103A antibody are provided below.

### Surface Plasmon Resonance Binding Assessments

The 5A7-N103A antibody binds to mouse LTBR with a K_{D} of 41+/−1.5 nM which is similar to the K_{D} of 1E01 binding to human LTBR (K_{D} of 115+/−26 nM). In addition, the on/off rates of the 5A7-N103A and 1E01 to mouse LTBR or human LTBR, respectively, were also similar.

### Epitope Determination and Structural Modeling

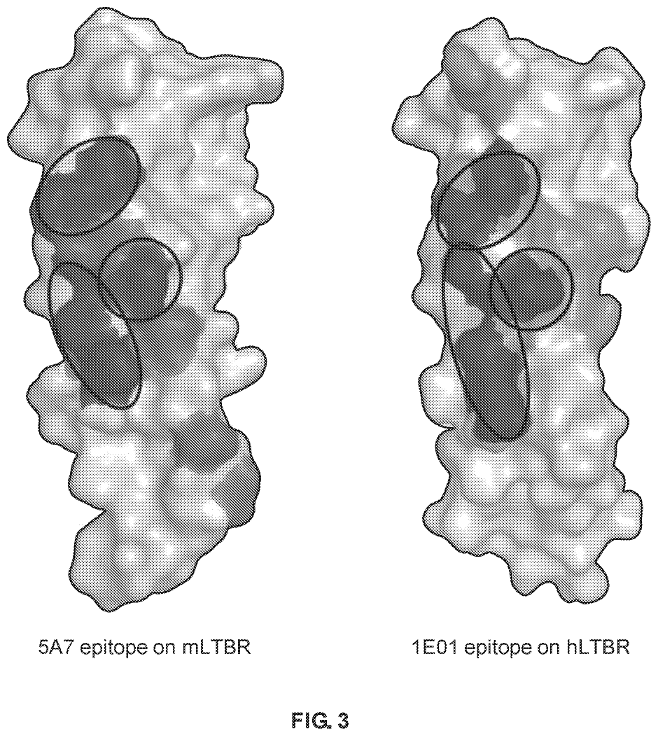

Crystallography studies were performed to evaluate the epitope binding of the 5A7 antibody (the parental Fab of the 5A7-N103A tetravalent antibody). These studies showed that the binding epitopes of 1E01 for human LTBR and 5A7 for mouse LTBR partially overlap. FIG. 3 depicts a surface representation of the binding epitope of 5A7 on mouse LTBR (FIG. 3, left side) and 1E01 on human LTBR (FIG. 3, right side). Residues of mouse LTBR or human LTBR participating in the interaction interface with 5A7 or 1E01 are shaded on the left or right side model, respectively. Residues of the shared epitope between mouse and human LTBR involving in binding to 5A7 and 1E01, respectively, are circled.

Structural modeling was performed to predict the binding geometry of the 1E01 and 5A7 Fabs to their respective human or mouse LTBR, with coaxial orientation of the Fabs to the cell membrane. Based on this modeling, the distance between the closest clustering of LTBR C-termini for the 1E01 tetravalent antibody and the 5A7-N103A tetravalent antibody was predicted to be similar, ranging from 110 angstroms to 120 angstroms for both 1E01 and 5A7-N103A-mediated clustering. Put another way, it is predicted that the 1E01 tetravalent antibody and the 5A7-N103A tetravalent antibody can each cluster 2 human or mouse LTBR molecules, respectively, in a cell membrane such that the 2 LTBR molecules are within 110-120 angstroms of each other. (This description is providing the relationship of the two closest LTBR molecules in a cell membrane bound by the 1E01 tetravalent antibody and the 5A7-N103A tetravalent antibody; as described elsewhere herein, each of these antibodies can bind up to four LTBR molecules—e.g. as shown in FIG. 1C).

Neither the 1E01 tetravalent antibody nor the 5A7-N103A tetravalent antibody block ligand binding to LTBR.

### Cell-Based Binding and Functional Assays

Tetravalent 1E01 and 5A7-N103A antibodies were characterized in a panel of cell-based binding and functional assays, which are described below and summarized in Table 5.

Endogenous human LTBR binding: 1E01 binding to endogenous LTBR on human endothelial cells (human umbilical vein endothelial cells (HUVEC)) and tumor cells (HT29 human colorectal carcinoma cells) was assessed in a flow cytometry-based assay using AF647-labeled antibody and Quantbrite™ beads. Cells were incubated with antibody for 1.5 hours on ice prior to endpoint analysis. Results confirmed the binding of 1E01 to endogenous human LTBR.

NFkB activation: 1E01 and 5A7-N103A-induced NFkB activation was determined in recombinant CT26 mouse endogenous LTBR negative NFkB-luciferase reporter cell lines stably expressing either a fully murine LTBR (“CT26 mEC-mTM-mIC NFkB”) or a chimeric LTBR with a human extracellular domain and mouse transmembrane and intracellular domains (“CT26 hEC-mTM-mIC NFkB”). This approach allowed for direct comparison of biological activity induced by 1E01 versus 5A7-N103A antibodies. Cells were incubated with different concentrations of antibody for 6 hours at 37° C. prior endpoint analysis. The results confirmed the restricted specificity, dose-dependent NFkB induction, and similar potency of tetravalent 1E01 and 5A7-N103A.

Cxcl10 release: 1E01 and 5A7-N103A-mediated induction of CXCL10 was assessed in primary human and cynomolgus monkey fibroblasts and a mouse NIH3T3 fibroblast cell line, respectively. Cells were incubated with different concentrations of antibody for 48 hours at 37° C. prior to endpoint analysis. Results demonstrated a dose-dependent secretion of CXCL10 for 1E01 and 5A7-N103A and similar potency of 1E01 in human and cynomolgus fibroblasts.

TABLE 5

| | | Antibody | |
|---|---|---|---|
| Assay | Cell Line | 1E01 tetravalent | 5A7-N103A tetravalent |
| Endogenous human | HUVEC | EC₅₀ (ug/mL): | Not Tested |
| LTβR Binding | | 0.076 | |
| | HT29 | EC₅₀ (ug/mL): | Not Tested |
| | | 0.073 | |
| NFkB Activation | CT26 hEC-mTM-mIC | EC₅₀ (ug/mL): | Not detected |
| | NFkB | 1.73 × 10⁻³ ± 9.82 × 10⁻⁴ | |
| | | (n = 3) | |
| | CT26 mEC-mTM-mIC | Not detected | EC₅₀ (ug/mL): |
| | NFkB | | 3.53 × 10⁻³ ± 1.32 × 10⁻³ |
| | | | (n = 2) |
| CXCL10 Release | Primary human | EC₅₀ (ug/mL): | Not Tested |
| | dermal fibroblasts | 2.31 × 10⁻³ | |
| | Primary cyno | EC₅₀ (ug/mL): | Not Tested |
| | fibroblasts | 9.02 × 10⁻³ | |
| | Mouse NIH3T3 | Not Tested | EC₅₀ (ug/mL): |
| | embryonic fibroblast | | 5.66 × 10⁻³ ± 3.62 × 10⁻³ |
| | cell line | | (n = 2) |

### In Vivo Studies

### CT26 Tumor Cells

5×10⁵ CT26 cells (murine colorectal carcinoma) were implanted subcutaneously into Balb/c mice. Post implantation, tumors were staged with average volumes of 50-100 mm³ and randomized prior to initiation of dosing 10 days post-implant. Mice were treated subcutaneously (SC) every 3 days for 3 doses (Q3Dx3) with one of three different types of tetravalent antibody: A) 5A7-N103A at increasing doses from 0.1-20 mg/kg; B) 20 mg/kg clone “5H7” anti-mouse LTBR (5H7 is a positive control antibody; it also has a similar affinity and similar activity in an NFkB activation assay to 1E01, but it binds to a different epitope than 1E01); C) 20 mg/kg isotype tetravalent control (negative control). Tumor volumes were measured twice a week until the end of the study, when the first mouse reached a tumor volume of 2250 mm³. N=9 or 10 mice per group. The results of the study are shown in Table 6.

TABLE 6

| | | | % TGI VS, | | | |
|---|---|---|---|---|---|---|
| | | | isotype | P-value | Average | |
| | | | control | (D22 | Body | |
| | | | (D22 post | post | Weight | |
| | Dose | Schedule | tumor | tumor | (BW) % | |
| Group | mg/kg | SC dosing | implant) | implant) | change | Number |
| Isotype | 20 | Q3D × 3 | — | — | 7.7 | 10 |
| (tetrafab) | | | | | | |
| 5H7 | 20 | Q3D × 3 | 59.5 | 0.0048 | −9.2 | 10 |
| 5A7-N103A | 20 | Q3D × 3 | 72.7 | 0.0010 | −8.7 | 10 |
| 5A7-N103A | 5 | Q3D × 3 | 57.4 | 0.0087 | −4.4 | 9 |
| 5A7-N103A | 1 | Q3D × 3 | 42.2 | 0.0861 | 6.6 | 10 |
| 5A7-N103A | 0.5 | Q3D × 3 | 27.3 | 0.8568 | 6.5 | 10 |
| 5A7-N103A | 0.1 | Q3D × 3 | 23.4 | 0.8233 | −1.0 | 10 |

In this study, treatment with 5 and 20 mg/kg of 5A7-N103A induced significant tumor growth inhibition (TGI) (57.4% and 72.7%, respectively). Whereas no significant TGI was evident at 0.1, 0.5, and 1 mg/kg doses of 5A7-N103, an overall trend of a dose response was observed. Body weight loss (BWL) was seen in 5 and 20 mg/kg 5A7-N103A groups, but it did not reach criteria for humane euthanasia (i.e. >20%). The positive control antibody (5H7) induced significant TGI (59.5%), with some BWL.

% TGI vs isotype control was calculated at the end of the study (D22 post-tumor implant) when the first mouse reached a tumor volume of 2250 mm3. Statistical analysis of TGI was conducted by ANCOVA. % change in body weight from day 10 (initiation of dosing) was also calculated at the end of the study.

### B16F10 Tumor Cells

In these studies, B16F10 (murine melanoma) tumor cells were used. B16F10 are PD(L)1 inhibitor-treatment resistant, and are an immunologically cold tumor model. (“Cold” tumors are non-inflamed and T-cell excluded, they have low/no responsiveness to immune modulators such as PD(L)1 inhibitors. In contrast, “hot” tumors have a higher level of T-cell infiltrates and proinflammatory cytokines; they have relatively high responsiveness to PD(L)1 inhibitors.)

5×10⁵ B16F10 cells were implanted subcutaneously into C57BL/6 mice. Post implantation, tumors were staged with average volumes of 50-100 mm³ and randomized prior to initiation of dosing 8 days post-implant. Mice were treated subcutaneously (SC) every 3 days for 3 doses (Q3Dx3) with one of three different types of tetravalent antibody: A) 5A7-N103A at increasing doses from 1-50 mg/kg; B) 20 mg/kg clone “5H7” anti-mouse LTBR; C) 50 mg/kg isotype control (negative control). In the same study, an alternative weekly dosing regimen of 5A7-N103A at 20 mg/kg or 50 mg/kg for 2 doses (QWx2) was also tested. Tumor volumes were measured twice a week until the end of the study, when the first mouse reached a tumor volume of 2250 mm³. Number (N)=7-9 mice per group. % TGI vs. isotype control was calculated at the end of the study (D18 post-tumor implant) when the first mouse reached a tumor of 2250 mm3. Statistical analysis of TGI was conducted by ANCOVA. % change in BW from day 10 (initiation of dosing) was also calculated at the end of the study. The results of the study are shown in Table 7.

TABLE 7

| | | % TGI | P-value vs | P-value | Average | Individual | |
|---|---|---|---|---|---|---|---|
| | Dose | (D18 post | isotype | vs. 5H7 | % BW | MBL | |
| Group | mg/kg | implant) | (Ancova) | (Ancova) | change | (%) | N |
| Isotype | 50 | — | — | — | 8.5 | — | 8 |
| tetravalent | | | | | | | |
| Q3D × 3 | | | | | | | |
| 5H7 | 20 | 45 | 0.08 | — | 0.8 | 4 | 7 |
| Q3D × 3 | | | | | | | |
| 5A7- | 50 | 67 | 0.0009 | 0.87 | −6.3 | −12.9 | 9 |
| N103A | | | | | | | |
| Q3D × 3 | | | | | | | |
| 5A7- | 20 | 51 | 0.048 | >0.99 | −8 | −20.3 | 9 |
| N103A | | | | | | | |
| Q3D × 3 | | | | | | | |
| 5A7- | 10 | 20 | 0.99 | 0.957 | −3.1 | −15.8 | 9 |
| N103A | | | | | | | |
| Q3D × 3 | | | | | | | |
| 5A7- | 5 | 22 | 0.99 | 0.99 | −0.6 | −9.5 | 7 |
| N103A | | | | | | | |
| Q3D × 3 | | | | | | | |
| 5A7- | 1 | 7 | >0.99 | 0.77 | 6.8 | −14.5 | 9 |
| N103A | | | | | | | |
| Q3D × 3 | | | | | | | |
| 5A7- | 50 | 63 | 0.0061 | 0.98 | −5.8 | −21.4 | 10 |
| N103A | | | | | | | |
| QW × 2 | | | | | | | |
| 5A7- | 20 | 58 | 0.0012 | 0.99 | −0.1 | −10 | 9 |
| N103A | | | | | | | |
| QW × 2 | | | | | | | |

In this study, treatment with 20 and 50 mg/kg of 5A7-N103A administered SC Q3Dx3 induced significant tumor growth inhibition (TGI) (51% and 67%, respectively). There was no significant efficacy observed at 1, 5, and 10 mg/kg doses of 5A7-N103, although there was an overall trend towards a dose-dependent response. Body weight loss (BWL) was seen in 10, and 50 mg/kg 5A7-N103A groups, but it did not reach criteria for humane euthanasia (i.e. >20%). The positive control antibody (5H7) (20 mg/kg) induced some TGI (45%), with some BWL.

For the QWx2 5A7-N103A group, significant TGI was evident at the 20 mg/kg and 50 mg/kg doses (58% and 63% TGI, respectively). BWL was observed in the 50 mg/kg group, but did not reach criteria for humane euthanasia.

In another related study the route of administration of 5A7-N103A was assessed. In the B16F10 model as above, 5A7-N103A was administered via subcutaneous (SC) or intravenous (IV) route QWx2 at 50 mg/kg. In this study, the SC and IV routes had very similar TGI, of 69% and 71% respectively. Also, at the endpoint of the study (day 18), the amount of intratumoral CD8+ T cells was measured in mice treated with isotype control and 5A7-N103A. There were 5.4-fold more intratumoral CD8+ T cells in the mice treated with 5A7-N103A as compared to those treated with isotype control.

Thus, these studies shows that 5A7-N103A promotes CD8+ T cell infiltration and tumor growth inhibition in a PD(L)1 inhibitor-resistant tumor model (B16F10 cells).

In another related study the effect of 5A7-N103A on CXCL13 and TCF-1 positive, CD8 positive T stem like cells (Tscm) was compared to that of an isotype antibody. CXCL13 is an important indicator of TLS induction, and TCF-1+, CD8+ Tscm give rise to functional effector cells. In the B16F10 model as above, 5A7-N103A was administered via IV route QWx2 at 50 mg/kg; an isotype antibody was also administered. In this study, 5A7-N103A resulted in over 2000 pg/mg CXCL13 (as compared to ˜0 pg/mg with the isotype antibody), and at least 4-fold greater number of TCF-1+, CD8+ Tscm cells as compared to the isotype antibody. Thus, 5A7-N103A also increases CXCL13 production and the presence of TCF-1+, CD8+ Tscm cells in this cold tumor model system (B16F10 cells).

### Additional Tumor Cell Lines

Single agent efficacy of 50 mg/kg 5A7-N103A administered SC QWx2 was also evaluated in several additional models and induced significant TGI as follows: MC38 (murine colon adenocarcinoma): 62% TGI; AT3 (mouse breast tumor): 63% TGI; MB49 (mouse urothelial tumor): 67% TGI; and KP 787 (mouse NSCLC): 72%.

Thus, 5A7-N103A induced significant TGI in multiple different tumor cell lines that represent diverse tumor microenvironments in terms of the composition and magnitude of immune infiltrates (i.e. inflamed vs. non-inflamed) and responsiveness to anti-PD(L)1 inhibitors.

### Combination Treatments

To investigate the potential for combinatorial efficacy, studies were performed using 5A7-N103A dosed in combination with or without an anti-PD1 antibody (internal clone F2) and with or without an anti-VEGF antibody (internal clone G6-31).

In the B16F10 model, the triple combination of 5A7-N103A+anti-PD-1+anti-VEGF induced significant TGI (87%) vs. isotype control, in addition to achieving significance over the single agent treatments (including 5A7-N103A induced TGI of 72%).

In the MB49 model, the triple combination induced significant TGI (96%) vs. isotype. This efficacy was also significant versus single agent treatments, including 5A7-N103A which induced 67% TGI in this model.

### CD8+ T Cell Depletion Study

To examine the mechanism of action of LTBR agonism-induced antitumor efficacy, a CD8+ T cell depletion study was conducted.

5×10⁵ MC38 cells were implanted subcutaneously into C57BL/6 mice. Post implantation, tumors were staged with average volumes of 50-100 mm3 and randomized prior to initiation of dosing on day 9 post implant. Different groups of mice were treated with the following different agents or combination of agents: A) 5A7-N103A; B) 5A7-N103A+anti-CD8 antibody; C) 5H7; D) 5H7+anti-CD8 antibody; E) isotype control. In this study, the 5A7-N103A was administered IV at 50 mg/kg weekly for 2 doses (QWx2), 5H7 was administered SC at 20 mg/kg every 3 days for 3 doses (Q3Dx3) and the anti-CD8 antibody was administered 200 micrograms twice a week.

In this study, 5A7-N103A alone and 5H7 alone induced significant TGI (5A7-N103A: 66% vs. isotype control; 5H7: 49% vs. isotype control). In contrast, there was no significant TGI in the 5A7-N103A in combination with anti-CD8 antibody or 5H7 in combination with anti-CD8 antibody (i.e. CD8 T cell-depleted) treated mice.

This shows the role of CD8+ T cells in the antitumor efficacy of anti-LTBR tetravalent 5A7-N103A and 5H7 antibodies.

### Summary of Sequences

Sequences provided in this application are summarized in Table 8 below.

TABLE 8

| SEQ | | |
|---|---|---|
| ID | | |
| NO: | Description | Sequence |
| 1 | 1E01 VH CDR1 - | GGTFSSY |
| | Chothia | |
| 2 | 1E01 VH CDR1 - | SYFWS |
| | Kabat | |
| 3 | 1E01 VH CDR1 - | GGTFSSYFWS |
| | Extended | |
| 4 | 1E01 VH CDR2 - | YSGGS |
| | Chothia | |
| 5 | 1E01 VH CDR2 - | RFYSGGSANYNPSLKE |
| | Kabat | |
| 6 | 1E01 VH CDR3 | ERRGYSGGFEI |
| 7 | 1E01 VL CDR1 | RSSQSLLHSNSYNYLD |
| 8 | 1E01 VL CDR2 | LGSYRAS |
| 9 | 1E01 VL CDR3 | MQPLQTPFT |
| 10 | 1E01 VH | QVQLVQSGAEVKKPGSSVKVSCKASGGTFSSYFWSWVRQAPGQGLEWMG |
| | | RFYSGGSANYNPSLKERVTITADESTSTAYMELSSLRSEDTAVYYCARE |
| | | RRGYSGGFEIWGQGTLVTVSS |
| 11 | 1E01 VL | DIVMTQSPLSLPVTPGEPASISCRSSQSLLHSNSYNYLDWYLQKPGQSP |
| | | QLLIYLGSYRASGVPDRFSGSGSGTDFTLKISRVEAEDVGVYYCMQPLQ |
| | | TPFTFGPGTKVDIK |
| 12 | 1E01 | QVQLVQSGAEVKKPGSSVKVSCKASGGTFSSYFWSWVRQAPGQGLEWMG |
| | Tetravalent | RFYSGGSANYNPSLKERVTITADESTSTAYMELSSLRSEDTAVYYCARE |
| | Heavy Chain | RRGYSGGFEIWGQGTLVTVSSASTKGPSVFPLAPSSKSTSGGTAALGCL |
| | | VKDYFPEPVTVSWNSGALTSGVHTFPAVLQSSGLYSLSSVVTVPSSSLG |
| | | TQTYICNVNHKPSNTKVDKKVEPKSCGGGGSQVQLVQSGAEVKKPGSSV |
| | | KVSCKASGGTFSSYFWSWVRQAPGQGLEWMGRFYSGGSANYNPSLKERV |
| | | TITADESTSTAYMELSSLRSEDTAVYYCARERRGYSGGFEIWGQGTLVT |
| | | VSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVD |
| | | KKVEPKSCDKTHTCPPCPAPEAAGAPSVFLFPPKPKDTLMISRTPEVTC |
| | | VVVDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 13 | 1E01 Light | DIVMTQSPLSLPVTPGEPASISCRSSQSLLHSNSYNYLDWYLQKPGQSP |
| | chain | QLLIYLGSYRASGVPDRFSGSGSGTDFTLKISRVEAEDVGVYYCMQPLQ |
| | | TPFTFGPGTKVDIKRTVAAPSVFIFPPSDEQLKSGTASVVCLLNNFYPR |
| | | EAKVQWKVDNALQSGNSQESVTEQDSKDSTYSLSSTLTLSKADYEKHKV |
| | | YACEVTHQGLSSPVTKSFNRGEC |
| 14 | 1E01 Bivalent | QVQLVQSGAEVKKPGSSVKVSCKASGGTFSSYFWSWVRQAPGQGLEWMG |
| | Heavy Chain | RFYSGGSANYNPSLKERVTITADESTSTAYMELSSLRSEDTAVYYCARE |
| | | RRGYSGGFEIWGQGTLVTVSSASTKGPSVFPLAPSSKSTSGGTAALGCL |
| | | VKDYFPEPVTVSWNSGALTSGVHTFPAVLOSSGLYSLSSVVTVPSSSLG |
| | | TQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPGK |
| 15 | Human LTBR, | MLLPWATSAPGLAWGPLVLGLFGLLAASQPQAVPPYASENQTCRDQEKE |
| | isoform 1 | YYEPQHRICCSRCPPGTYVSAKCSRIRDTVCATCAENSYNEHWNYLTIC |
| | (UniProtKB | QLCRPCDPVMGLEEIAPCTSKRKTQCRCQPGMFCAAWALECTHCELLSD |
| | P36941) | CPPGTEAELKDEVGKGNNHCVPCKAGHFQNTSSPSARCQPHTRCENQGL |
| | | VEAAPGTAQSDTTCKNPLEPLPPEMSGTMLMLAVLLPLAFFLLLATVFS |
| | | CIWKSHPSLCRKLGSLLKRRPQGEGPNPVAGSWEPPKAHPYFPDLVQPL |
| | | LPISGDVSPVSTGLPAAPVLEAGVPQQQSPLDLTREPQLEPGEQSQVAH |
| | | GTNGIHVTGGSMTITGNIYIYNGPVLGGPPGPGDLPATPEPPYPIPEEG |
| | | DPGPPGLSTPHQEDGKAWHLAETEHCGATPSNRGPRNQFITHD |
| 16 | Mouse LTBR | MRLPRASSPCGLAWGPLLLGLSGLLVASQPQLVPPYRIENQTCWDQDKE |
| | (UniProtKB - | YYEPMHDVCCSRCPPGEFVFAVCSRSQDTVCKTCPHNSYNEHWNHLSTC |
| | P50284) | QLCRPCDIVLGFEEVAPCTSDRKAECRCQPGMSCVYLDNECVHCEEERL |
| | | VLCQPGTEAEVTDEIMDTDVNCVPCKPGHFQNTSSPRARCQPHTRCEIQ |
| | | GLVEAAPGTSYSDTICKNPPEPGAMLLLAILLSLVLFLLFTTVLACAWM |
| | | RHPSLCRKLGTLLKRHPEGEESPPCPAPRADPHFPDLAEPLLPMSGDLS |
| | | PSPAGPPTAPSLEEVVLQQQSPLVQARELEAEPGEHGQVAHGANGIHVT |
| | | GGSVTVTGNIYIYNGPVLGGTRGPGDPPAPPEPPYPTPEEGAPGPSELS |
| | | TPYQEDGKAWHLAETETLGCQDL |
| 17 | Cynomolgus | MRLPWATSAPGLAWGPLVLGLFGLLAASQPQVVRKGPVPPYGSENQTCR |
| | monkey LTBR | DQEKEYYEPRHRICCSRCPPGTYVSAKCSRSRDTVCATCAENSYNEHWN |
| | (UniProtKB - | YLTICQLCRPCDPVMGLEEIAPCTSKRKTQCRCQPGMFCAAWALECTHC |
| | A0A2K5VGQ6) | ELLSDCPPGTEAELKDEVGKGNNHCVPCKAGHFONTSSPSARCQPHTRC |
| | | EDQGLVEAAPGTAQSDTTCRNPSESLPPEMSGTMLMLAILLPLAFFLLL |
| | | ATIFACIWKSHPSLCRKLGSLLKRHPQGEGPNPVAAGRDPPKANPQYPD |
| | | LVEPLLPISGDVSPVSTGLPTALVSEEGVPQQQSPLDLTTEPQLEPGEQ |
| | | NQVAHGTNGIHVTGGSMTITGNIYIYNGPVLGGPPGPGDLPATPDPPYP |
| | | IPEEGDPGPPGLSTPHQEDGKAWHLAETEHCGATPSNRGPRSQFITYD |
| 18 | Linker | EPKSCGGGGS |
| 19 | Polynucleotide | CAAGTGCAGCTTGTGCAGAGCGGAGCCGAAGTCAAGAAGCCCGGGTCGT |
| | encoding 1E01 | CAGTGAAAGTGTCCTGCAAGGCCTCCGGCGGAACCTTCAGCTCGTACTT |
| | tetravalent | CTGGTCCTGGGTTAGGCAGGCCCCTGGACAAGGCCTCGAATGGATGGGT |
| | heavy chain | CGGTTCTACTCCGGCGGTTCCGCCAACTACAACCCGTCACTGAAGGAGA |
| | (encoding SEQ | GAGTGACCATTACCGCGGATGAGTCGACTAGCACCGCCTACATGGAACT |
| | ID NO: 12) | GTCCAGCCTGCGGTCCGAGGACACTGCAGTCTACTATTGTGCTCGCGAG |
| | | CGGCGCGGGTACTCTGGCGGATTTGAAATCTGGGGACAGGGAACCCTGG |
| | | TCACCGTCTCCTCAGCCTCCACCAAGGGCCCATCGGTCTTCCCCCTGGC |
| | | ACCCTCCTCCAAGAGCACCTCTGGGGGCACAGCGGCCCTGGGCTGCCTG |
| | | GTCAAGGACTACTTCCCCGAACCGGTGACGGTGTCGTGGAACTCAGGCG |
| | | CCCTGACCAGCGGCGTGCACACCTTCCCGGCTGTCCTACAGTCCTCAGG |
| | | ACTCTACTCCCTCAGCAGCGTGGTGACCGTGCCCTCCAGCAGCTTGGGC |
| | | ACCCAGACCTACATCTGCAACGTGAATCACAAGCCCAGCAACACCAAGG |
| | | TGGACAAGAAAGTTGAGCCTAAGAGCTGCGGTGGCGGTGGATCACAAGT |
| | | GCAGCTTGTGCAGAGCGGAGCCGAAGTCAAGAAGCCCGGGTCGTCAGTG |
| | | AAAGTGTCCTGCAAGGCCTCCGGCGGAACCTTCAGCTCGTACTTCTGGT |
| | | CCTGGGTTAGGCAGGCCCCTGGACAAGGCCTCGAATGGATGGGTCGGTT |
| | | CTACTCCGGCGGTTCCGCCAACTACAACCCGTCACTGAAGGAGAGAGTG |
| | | ACCATTACCGCGGATGAGTCGACTAGCACCGCCTACATGGAACTGTCCA |
| | | GCCTGCGGTCCGAGGACACTGCAGTCTACTATTGTGCTCGCGAGCGGCG |
| | | CGGGTACTCTGGCGGATTTGAAATCTGGGGACAGGGAACCCTGGTCACC |
| | | GTCTCCTCAGCCTCCACCAAGGGCCCATCGGTCTTCCCCCTGGCACCCT |
| | | CCTCCAAGAGCACCTCTGGGGGCACAGCGGCCCTGGGCTGCCTGGTCAA |
| | | GGACTACTTCCCCGAACCGGTGACGGTGTCGTGGAACTCAGGCGCCCTG |
| | | ACCAGCGGCGTGCACACCTTCCCGGCTGTCCTACAGTCCTCAGGACTCT |
| | | ACTCCCTCAGCAGCGTGGTGACCGTGCCCTCCAGCAGCTTGGGCACCCA |
| | | GACCTACATCTGCAACGTGAATCACAAGCCCAGCAACACCAAGGTGGAC |
| | | AAGAAAGTTGAGCCCAAATCTTGTGACAAAACTCACACATGCCCACCGT |
| | | GCCCAGCACCTGAAGCCGCTGGGGCACCGTCAGTCTTCCTCTTCCCCCC |
| | | AAAACCCAAGGACACCCTCATGATCTCCCGGACCCCTGAGGTCACATGC |
| | | GTGGTGGTGGACGTGAGCCACGAAGACCCTGAGGTCAAGTTCAACTGGT |
| | | ACGTGGACGGCGTGGAGGTGCATAATGCCAAGACAAAGCCGCGGGAGGA |
| | | GCAGTACAACAGCACGTACCGTGTGGTCAGCGTCCTCACCGTCCTGCAC |
| | | CAGGACTGGCTGAATGGCAAGGAGTACAAGTGCAAGGTCTCCAACAAAG |
| | | CCCTCCCAGCCCCCATCGAGAAAACCATCTCCAAAGCCAAAGGGCAGCC |
| | | CCGAGAACCACAGGTGTACACCCTGCCCCCATCCCGGGAGGAGATGACC |
| | | AAGAACCAGGTCAGCCTGACCTGCCTGGTCAAAGGCTTCTATCCCAGCG |
| | | ACATCGCCGTGGAGTGGGAGAGCAATGGGCAGCCGGAGAACAACTACAA |
| | | GACCACGCCTCCCGTGCTGGACTCCGACGGCTCCTTCTTCCTCTATAGC |
| | | AAGCTCACCGTGGACAAGAGCAGGTGGCAGCAGGGGAACGTCTTCTCAT |
| | | GCTCCGTGATGCATGAGGCTCTGCACAACCACTACACGCAGAAGAGCCT |
| | | CTCCCTGTCCCCCGGAAAA |
| 20 | Polynucleotide | GACATTGTGATGACTCAGTCGCCGTTGTCGCTGCCTGTGACTCCTGGAG |
| | encoding 1E01 | AGCCGGCCTCGATCTCCTGCCGGTCAAGCCAGTCCCTGCTGCACTCCAA |
| | light chain | CTCCTATAACTACCTGGATTGGTACCTCCAAAAGCCTGGGCAGAGCCCC |
| | (encoding SEQ | CAGCTCCTGATCTACCTTGGCTCTTACCGGGCCTCCGGAGTGCCGGACA |
| | ID NO: 13) | GATTCAGCGGTTCCGGATCAGGAACCGACTTTACCCTGAAAATCTCCCG |
| | | CGTGGAAGCGGAAGATGTCGGCGTGTACTACTGTATGCAGCCCCTGCAA |
| | | ACTCCATTCACCTTCGGGCCCGGCACCAAGGTCGACATCAAACGAACTG |
| | | TGGCTGCACCATCTGTCTTCATCTTCCCGCCATCTGATGAGCAGTTGAA |
| | | ATCTGGAACTGCCTCTGTTGTGTGCCTGCTGAATAACTTCTATCCCAGA |
| | | GAGGCCAAAGTACAGTGGAAGGTGGATAACGCCCTCCAATCGGGTAACT |
| | | CCCAGGAGAGTGTCACAGAGCAGGACAGCAAGGACAGCACCTACAGCCT |
| | | CAGCAGCACCCTGACGCTGAGCAAAGCAGACTACGAGAAACACAAAGTC |
| | | TACGCCTGCGAAGTCACCCATCAGGGCCTGAGCTCGCCCGTCACAAAGA |
| | | GCTTCAACAGGGGAGAGTGT |
| 21 | Polynucleotide | CAAGTGCAGCTTGTGCAGAGCGGAGCCGAAGTCAAGAAGCCCGGGTCGT |
| | encoding VH | CAGTGAAAGTGTCCTGCAAGGCCTCCGGCGGAACCTTCAGCTCGTACTT |
| | 1E01 (encoding | CTGGTCCTGGGTTAGGCAGGCCCCTGGACAAGGCCTCGAATGGATGGGT |
| | SEQ ID NO: | CGGTTCTACTCCGGCGGTTCCGCCAACTACAACCCGTCACTGAAGGAGA |
| | 10) | GAGTGACCATTACCGCGGATGAGTCGACTAGCACCGCCTACATGGAACT |
| | | GTCCAGCCTGCGGTCCGAGGACACTGCAGTCTACTATTGTGCTCGCGAG |
| | | CGGCGCGGGTACTCTGGCGGATTTGAAATCTGGGGACAGGGAACCCTGG |
| | | TCACCGTCTCCTCA |
| 22 | Polynucleotide | GACATTGTGATGACTCAGTCGCCGTTGTCGCTGCCTGTGACTCCTGGAG |
| | encoding VL | AGCCGGCCTCGATCTCCTGCCGGTCAAGCCAGTCCCTGCTGCACTCCAA |
| | 1E01 | CTCCTATAACTACCTGGATTGGTACCTCCAAAAGCCTGGGCAGAGCCCC |
| | (encoding SEQ | CAGCTCCTGATCTACCTTGGCTCTTACCGGGCCTCCGGAGTGCCGGACA |
| | ID NO: 11) | GATTCAGCGGTTCCGGATCAGGAACCGACTTTACCCTGAAAATCTCCCG |
| | | CGTGGAAGCGGAAGATGTCGGCGTGTACTACTGTATGCAGCCCCTGCAA |
| | | ACTCCATTCACCTTCGGGCCCGGCACCAAGGTCGACATCAAA |

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting. The term “or” when used in the context of a listing of multiple options (e.g. “A, B, or C”) shall be interpreted to include any one or more of the options, unless the context clearly dictates otherwise.

Exemplary methods and materials are described herein, although methods and materials similar or equivalent to those described herein can also be used in the practice or testing of the present invention. The materials, methods, and examples are illustrative only and not intended to be limiting.

It is claimed:

1. An isolated antibody that binds to lymphotoxin beta receptor (LTBR) and comprises a heavy chain variable region (VH) and a light chain variable region (VL), wherein
the VH complementarity determining region (CDR) one comprises the amino acid sequence of SEQ ID NOs: 1, 2, or 3,
the VH CDR2 comprises the amino acid sequence of SEQ ID NOs: 4 or 5,
the VH CDR3 comprises the amino acid sequence of SEQ ID NO: 6,
the VL CDR1 comprises the amino acid sequence of SEQ ID NO: 7,
the VL CDR2 comprises the amino acid sequence of SEQ ID NO: 8, and
the VL CDR3 comprises the amino acid sequence of SEQ ID NO: 9.

2. The antibody of claim 1, wherein the VH comprises the amino acid sequence of SEQ ID NO: 10 or a variant of SEQ ID NO: 10 thereof comprising one to four amino acid substitutions at residues that are not within a CDR, and the VL comprises the amino acid sequence of SEQ ID NO: 11 or a variant of SEQ ID NO: 11 thereof comprising one to four amino acid substitutions at residues that are not within a CDR.

3. The antibody of claim 2, wherein the VH comprises the amino acid sequence of SEQ ID NO: 10 and the VL comprises the amino acid sequence of SEQ ID NO: 11.

4-6. (canceled)

7. An isolated tetravalent antibody comprising a first antigen binding site, a second antigen binding site, a third antigen binding site, and a fourth antigen binding site, which each of the first, second, third, and fourth antigen binding sites bind to LTBR, and wherein each of the first, second, third, and fourth antigen binding sites comprise a VH and VL, and wherein for each of the first, second, third, and fourth antigen binding sites VH and VL:
the VH complementarity determining region (CDR) one comprises the amino acid sequence of SEQ ID NOs: 1, 2, or 3,
the VH CDR2 comprises the amino acid sequence of SEQ ID NOs: 4 or 5,
the VH CDR3 comprises the amino acid sequence of SEQ ID NO: 6,
the VL CDR1 comprises the amino acid sequence of SEQ ID NO: 7,
the VL CDR2 comprises the amino acid sequence of SEQ ID NO: 8, and
the VL CDR3 comprises the amino acid sequence of SEQ ID NO: 9.

8. The antibody of claim 7, wherein each of the first, second, third, and fourth VH comprises the amino acid sequence of SEQ ID NO: 10 or a variant of SEQ ID NO: 10 comprising one to four amino acid substitutions at residues that are not within a CDR, and each of the first, second, third, and fourth VL comprises the amino acid sequence of SEQ ID NO: 11 or a variant of SEQ ID NO: 11 thereof comprising one to four amino acid substitutions at residues that are not within a CDR.

9. The antibody of claim 8, wherein each of the first, second, third, and fourth VH comprises the amino acid sequence of SEQ ID NO: 10 and each of the first, second, third, and fourth VL comprises the amino acid sequence of SEQ ID NO: 11.

10. The antibody of claim 8, wherein the antibody comprises a light chain comprising the amino acid sequence of SEQ ID NO: 13 and a heavy chain comprising the amino acid sequence of SEQ ID NO: 12, wherein the C-terminal lysine of SEQ ID NO:12 is optional.

11. The antibody of claim 10, wherein the antibody comprises four light chains and two heavy chains.

12. The antibody of a claim 10, wherein the antibody is capable of simultaneously binding four separate LTBR molecules.

13. An isolated antibody that binds to LTBR, wherein the antibody binds to an epitope on LTBR comprising one or more amino acid residues selected from the group consisting of P63, P64, G65, T66, Y67, S69, A70, R76, T78, V79, C80, T82, C83, A84, E85, W92, K119 of SEQ ID NO: 15.

14. (canceled)

15. The antibody of claim 13, wherein the antibody is tetravalent and comprises a first antigen binding site, a second antigen binding site, a third antigen binding site, and a fourth antigen binding site, and wherein each of the first, second, third, and fourth antigen binding sites bind to the same epitope on four different LTBR molecules.

16. (canceled)

17. The antibody of claim 1, wherein the antibody mediates at least one of the following activities: (i) promotes the clustering of LTBR on the cell membrane; (ii) promotes LTBR-mediated NFkB activation; (iii) induces Cxcl10 secretion; (iv) induces IL-12 secretion; (v) inhibits tumor growth.

18. The antibody of claim 1, wherein the antibody does not block the binding of ligand to LTBR.

19. The antibody of claim 18, wherein the ligand is LIGHT or LTa1B2.

20. The antibody of claim 13, wherein the antibody comprises a heavy chain variable region (VH), and wherein the VH complementarity determining region (CDR) one comprises the amino acid sequence of SEQ ID NOs: 1, 2, or 3, the VH CDR2 comprises the amino acid sequence of SEQ ID NOs: 4 or 5, and the VH CDR3 comprises the amino acid sequence of SEQ ID NO: 6.

21. (canceled)

22. The antibody of claim 13, wherein the antibody comprises a VH that comprises the amino acid sequence of SEQ ID NO: 10 and the antibody comprises a VL that comprises the amino acid sequence of SEQ ID NO: 11.

23. The antibody of claim 1, wherein the antibody is tetravalent and has modifications in the Fc domain to reduce binding to the Fc gamma receptor.

24. An isolated polynucleotide or polynucleotides comprising one or more nucleotide sequences encoding the antibody of claim 1.

25. An isolated polynucleotide or polynucleotides comprising one or more nucleotide sequences encoding the VH, VL, or both of an antibody that binds LTBR, wherein the polynucleotide(s) comprise the VH nucleic acid sequence of SEQ ID NO: 21, the VL nucleic acid sequence of SEQ ID NO: 22, or both the VH nucleic acid sequence of SEQ ID NO: 21 and the VL nucleic acid sequence of SEQ ID NO: 22.

26. AA The isolated polynucleotide or polynucleotides of claim 25 comprising one or more nucleotide sequences encoding the heavy chain, light chain, or both of an antibody that binds LTBR, wherein the polynucleotide(s) comprise the heavy chain nucleic acid sequence of SEQ ID NO: 19, the light chain nucleic acid sequence of SEQ ID NO: 20, or both the heavy chain nucleic acid sequence of SEQ ID NO: 19 and the light chain nucleic acid sequence of SEQ ID NO: 20.

27. An isolated polynucleotide or polynucleotides comprising one or more nucleotide sequences encoding the VH, VL, or both of an antibody that binds LTBR, wherein the polynucleotide(s) comprise the VH nucleic acid sequence of the insert of the plasmid deposited with the ATCC and having ATCC Accession Number PTA-127515, the VL nucleic acid sequence of the insert of the plasmid deposited with the ATCC and having ATCC Accession Number PTA-127516, or both the VH nucleic acid sequence of the insert of the plasmid deposited with the ATCC and having ATCC Accession Number PTA-127515 and the VL nucleic acid sequence of the insert of the plasmid deposited with the ATCC and having ATCC Accession Number PTA-127516.

28-31. (canceled)

32. A vector comprising the polynucleotide or polynucleotides of any one of claim 24.

33. An isolated host cell comprising the vector of claim 32.

34. A method of producing an antibody, comprising culturing the host cell of claim 33 under conditions that result in production of the antibody, and recovering the antibody.

35. A pharmaceutical composition comprising a therapeutically effective amount of the antibody of claim 1 and a pharmaceutically acceptable carrier.

36. A method of treating cancer in a subject, the method comprising administering to a subject in need thereof a therapeutically effective amount of the antibody of claim 1.

37. The antibody of claim 1 for use a medicament

38-39. (canceled)

40. The use of the antibody of any one of claim 1 in the manufacture of a medicament for use in the treatment of cancer.

41. The method of claim 36, wherein the cancer is bladder cancer, breast cancer, clear cell kidney cancer, head/neck squamous cell carcinoma [squamous cell carcinoma of the head and neck (SCCHN)], lung squamous cell carcinoma, lung adenocarcinoma, malignant melanoma, non-small-cell lung cancer (NSCLC), ovarian cancer, pancreatic cancer, prostate cancer, renal cell carcinoma (RCC), small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), endometrial cancer, B-cell acute lymphoblastic leukemia, colorectal cancer (CRC), glioblastoma, uterine cancer, cervical cancer, penile cancer, gastric cancer (GC) or non-melanoma skin cancer.

42. The method of claim 41, wherein the cancer was previously treated with a PD1 or PDL1 [PD(L)1] inhibitor.
